(19) United States
(12) Patent Application Publication
(10) Pub. No.: US 2024/0425810 A1
(43) Pub. Date: Dec. 26, 2024

(54) CELLULAR SUPPORT FOR CULTURING METHODS

(71) Applicant: UNIVERSIDAD DEL PAÍS VASCO/EUSKAL HERRIKO UNIBERTSITATEA, Leioa - Vizcaya (ES)

(72) Inventors: Lourdes BASABE DESMONTS, Leioa - Vizcaya (ES); Fernando BENITO LÓPEZ, Leioa - Vizcaya (ES); Enrique AZUAJE HUALDE, Leioa - Vizcaya (ES); María de los Ángeles MARTÍNEZ DE PANCORBO GÓMEZ, Leioa - Vizcaya (ES); Yara ÁLVAREZ BRAÑA, Leioa - Vizcaya (ES)

(21) Appl. No.: 18/692,404

(22) PCT Filed: Sep. 16, 2022
(86) PCT No.: PCT/EP2022/075828

§ 371 Date: Mar. 15, 2024

(30) Foreign Application Priority Data
Sep. 17, 2021 (EP) 21382840.3

(51) Int. Cl. C12N 5/00

(52) U.S. Cl. CPC C12N 5/0075; C12N 2501/115; C12N 2501/165; C12N 2531/00; C12N 2533/30; C12N 2533/52; C12N 2535/10

(57) ABSTRACT

The inventors of the present invention have developed a support that couples the controlled seeding of cells into predetermined patterns with the presentation and/or detection of molecules of interest to/from said cells. Said support is characterized for containing predetermined patterns where cells adhere to and have a uniform layer of microbeads functionalized with a molecule of interest.

### TECHNICAL FIELD OF THE INVENTION

The present invention relates to the field of cellular biology and, more in particular, to assays for the study of the interaction of cells with the components of the extracellular matrix as well as for the analysis of compounds which are secreted by cells

### BACKGROUND OF THE INVENTION

In physiological conditions, cells forming multicellular organisms are confined within a dynamic and complex microenvironment, which is composed of the different cells sharing a functional grouping and the three dimensional structural network of macromolecules known as extracellular matrix (ECM). The interaction between cells and all the components of their microenvironments greatly affect and regulate cell function.

Conventional in vitro studies, which are usually carry out by seeding cells on cell adhesive substrates and incubating them with a cell culture medium, do not typically control the different interactions that may affect the cells. For instance, soluble signaling biomolecules such as growth factors are frequently diluted directly into the cells' medium, not taking into account the interactions of said molecules with the cells' microenvironment. In physiological conditions, the ECM functions as a reservoir for growth factors and regulates their presentation to the cells. This greatly improves efficiency of their interaction, since it not only localizes the growth factor to the proximity of the cell but also permits a crosstalk with other cell-ECM interactions that result in synergistic signaling.

In the lookout for novel platforms that allow tighter control and replication of cell microenvironments in vitro, research has focused on developing methodologies that allow the production of synthetic 3D ECMs and the study of the effect of it in the cell-adhesion, cell migration and cell differentiation processes among others at an in vitro level. These advances have also allowed the generation of novel platforms and biomaterials for the controlled delivery of drugs and soluble factors. This has branched into a wide variety of different technologies, including the development of 3D-like microenvironments based on biomaterials, the fabrication of functional tissue- and organ-on-chip microtechnologies and the generation of high-throughput cell analytical and monitoring systems with controlled cell distribution and cell-microenvironment interactions. In regards of soluble and growth factors presentations, research has centered on the development of biomaterials and systems for solid-phase presentation of growth factors, for potential applications on regenerative medicine and biological research.

However, while most of the new advances in these areas have produced promising outcomes for specific biological scenarios, there is still the need to generate technologies that allow the simple and versatile adaptation of cell microenvironments to different cellular and histological contexts with high control over biochemical and biomechanical cues and their monitoring. Developing a tunable technology that allows tight control over cells' microenvironments, specifically one that combines systems for solid-phase presentation of soluble and growth factors with control over cell localization, distribution and cell-cell contact, is a goal that has not been reached.

### SUMMARY OF THE INVENTION

The inventors of the present invention have developed a support that combines the controlled seeding of cells into predetermined patterns with the presentation and/or detection of molecules of interest to/from said cells.

Therefore, a first aspect of the present invention relates to a support suitable for culturing cells characterized in that it shows patterned regions, said pattern being defined by a complementary pattern of uniformly distributed microbeads attached to the support and wherein the microbeads are modified with at least one functionalizing molecule, wherein the patterned regions in the support defined by the microbeads show increased adhesive capacity to cells with respect to the regions to which the microbeads are attached.

In another aspect, the present invention relates to a method for producing the support according to the invention comprising the steps of:
(i) Providing a support;
(ii) Contacting the support with a stamp, said stamp having protrusions which define the desired pattern in the support, wherein the contact of the support with the stamp forms bottomless channels between the support and the stamp;
(iii) Contacting the support along the bottomless channels with a microbead suspension and allowing the microbeads to adhere to the support as to obtain a uniform deposition and attachment of the microbeads onto those regions in the support which are not protected by the protrusions of the stamp.

Yet another aspect of the present invention relates to a method for determining the effect of a molecule on a cell population which comprises the steps of:
(i) Providing a support according to the invention wherein said support contains the cell population wherein the cells of the population are attached to the support and wherein the molecule, the effect of which the on the cell population is to be determined, is the functionalizing molecule;
(ii) Maintaining the support under adequate conditions to allow the functionalizing molecule to exert its effect on the cells of the cell population; and
(iii) Detecting the response of the cells within the cell population to said functionalizing molecule,
wherein the detection of a response of the cells to the functionalizing molecule is indicative that the functionalizing molecule exerts an effect on the cells of the cell population.

Another aspect of the present invention relates to a method for determining if a cell population secretes to the medium a substance of interest which comprises the steps of:
(i) Providing a support according to the invention wherein said support contains a cell population wherein the cells of the population are attached to the support and wherein the functionalizing molecule comprises a molecule which is capable of interacting with the substance of interest, said interaction resulting in a detectable signal,
(ii) Culturing the cells in the adequate conditions to allow binding of the substances of interest secreted to the medium to the functionalizing molecule; and
(iii) Detecting the binding of the substance of interest to the molecule of interest by detecting the signal resulting from said interaction.

Another aspect of the present invention relates to a method for determining the effect of an effector molecule on a cell population and for determining if the cell population secretes to the medium a substance of interest which comprises the steps of:
(i) Providing a support according to any of claims 1 to 18 15 wherein said support contains the cell population, wherein the cells of the population are attached to the support and wherein the first and second type of microbeads contain, respectively, a first type of functionalizing molecule which is the effector molecule and a second type of functionalizing molecule which is capable of interacting with the substance of interest, said interaction resulting in a detectable signal;
(ii) Culturing the cells under conditions adequate to allow the effector molecule to exert its effect on the cells of the cell population and the binding of the substances of interest secreted to the medium to the detector molecule; and
(iii) Detecting the response of the cells within the cell population to said effector molecule and detecting the binding of the substance of interest to the detector molecule by detecting the signal resulting from said interaction.

### BRIEF DESCRIPTION OF THE FIGURES

FIG. 1. Combined array of hHF-MSCs and microbeads. A) Brightfield microscope images of the pattern of microbeads when loaded trough vacuum lithography (left) and capillarity (right). B) Plot of the array occupancy (number of dots occupied per array) at different times (30, 60 and 120 minutes) for the arrays of 50 and 100 μm. Error bars mean±SD (n=3). B) and C) represents brightfield microscope images of the occupied 50 and 100 μm dots and the arrays, respectively.

FIG. 2. Solid-phase presentation of FGF-2 to D50 hHF-MSCs arrays with co-patterned functionalized microbeads. A) Plots of the number of cells per array for the cells co-patterned in 50 μm dots with 0% and 50% FGF-2 functionalized microbeads. Error bars mean±SD (n=3). Statistical significance; paired two-tailed t-test (* p<0.05). B) Plots of the percentage of dot occupation (cells per dot) for the D50 cell patterns with 0% and 50% FGF-2 functionalized microbeads at 24 hours. C) Brightfield microscope images of the hHF-MSCs in arrays co-patterned with 0%, 50% or 100% FGF-2 functionalized microbeads.

FIG. 3. Solid-phase presentation of FGF-2 to D100 hHF-MSCs arrays with co-patterned functionalized microbeads. A) Plots of the number of cells per array for the cells co-patterned in 100 μm dots with 0% and 50% FGF-2 functionalized microbeads. Error bars mean±SD (n=3). Statistical significance; paired two-tailed t-test (** p<0.01). B) Plots of the percentage of dot occupation (cells per dot) for the D100 cell patterns with 0% and 50% FGF-2 functionalized microbeads at 24 hours. C) Brightfield microscope images of the hHF-MSCs in arrays co-patterned with 0%, 50% or 100% FGF-2 functionalized microbeads.

FIG. 4. FGFR-1 expression stimulation of hHF-MSCs in D100 arrays with co-patterned FGF-2 functionalized microbeads. A) Plot of the fluorescence intensity in the cell isles patterned with 0% and 50% FGF-2 functionalized microbeads, normalized to the mean fluorescence intensity of the 0% FGF-2 patterns. Each point represents a specific cell isle (n=3, 30 cells aisle per experimental condition). Statistical significance; paired two-tailed t-test (** p<0.001). B) Brightfield and fluorescence microscope images of the hHF-MSCs (top) and FGFR-1 expression (bottom) in arrays co-patterned with 0% or 50% FGF-2 functionalized microbeads.

FIG. 5. Solid-phase presentation of patterned hHF-MSCs on D₅₀ arrays co-patterned with 0% and 50% FGF-2 functionalized microbeads at 0 h. Top) Plot of number of cell per dot Bottom; Brightfield microscopy images of the cells.

FIG. 6. Solid-phase presentation of patterned hHF-MSCs on D₅₀ arrays co-patterned with 0% and 50% FGF-2 functionalized microbeads at 24 h. Top) Plot of number of cell per dot Bottom; Brightfield microscopy images of the cells.

FIG. 7. Solid-phase presentation of patterned hHF-MSCs on D₁₀₀ arrays co-patterned with 0% and 50% FGF-2 functionalized microbeads at 0 h. Top) Plot of number of cell per dot Bottom; Brightfield microscopy images of the cells.

FIG. 8. Solid-phase presentation of patterned hHF-MSCs on D₁₀₀ arrays co-patterned with 0% and 50% FGF-2 functionalized microbeads at 24 h. Top) Plot of number of cell per dot Bottom; Brightfield microscopy images of the cells.

FIG. 9. Stimulation of hHF-MSCs with diluted FGF-2. A) Plot of the number of cells per well when incubated for 48 hours in either serum free medium (SFM), SFM+FGF-2 (10 ng mL⁻¹), complete medium (CM) or CM+FGF-2 (10 ng mL⁻¹) Error bars mean±SD (n=3 samples per experimental condition). B) Brightfield microscope images of attached hHF-MSCs in the different conditions.

FIG. 10. Stimulation of hHF-MSCs with FGF-2 treated wells. A) Plot of the number of cells per well when incubated for 24 hours in non-treated wells with SFM, SFM+FGF2 (10 ng/ml) or in FGF-2 fixed to the bottom of the wells (SFM+FGF_{f}) with SFM. Error bars mean±SD (n=3 samples per experimental condition). B) Brightfield microscope images of attached hHF-MSCs in the different conditions.

FIG. 11. Stimulation of hHF-MSCs with FGF-2 functionalized microbeads. A) Plot of the number of cells per array when incubated for 24 hours on top of 0% and 100% FGF functionalized microbeads arrays. Error bars mean±SD (n=3 samples per experimental condition). B) Brightfield and fluorescence microscope images of attached hHF-MSCs in the different conditions. Cells in blue in the zoomed images indicates cell's nucleus.

FIG. 12. Detection of VEGF on patterned microbeads. A) Graphical representation of the detection moieties for the microbeads functionalized with VEGF-Ab and VEGF-SSSA. B) Plot of the normalized fluorescence intensity obtained from the incubation of different VEGF concentrations (0.001-1.000 μg mL⁻¹) with patterned microbeads functionalized with VEGF-Ab and VEGF-SSSA. Error bars correspond to the mean values±SD (n=3). C) Fluorescence microscopy images of the patterned microbeads functionalized with VEGF-Ab (top) and VEGF-SSSA (bottom) incubated with different concentrations of VEGF.

FIG. 13. A) Plot of the cell-secretion of VEGF from hHF-MSCs cultivated in SFM and CM quantified through ELISA. B) Plot of the normalized fluorescence intensity obtained from the incubation of hHF-MSCs supernatant (10⁵ cells per mL, 48 h) or CM medium (negative control, NC) with patterned microbeads functionalized with VEGF-Ab. C) Fluorescence microscopy images of the patterned microbeads functionalized with VEGF-Ab incubated with SFM, serving as negative control (top) and hHF-MSCs supernatant (bottom). Error bars correspond to the mean values±SD (n=3).

FIG. 14. Direct detection of the secretion of VEGF by combined patterns of small cell-colonies and microbeads functionalized with VEGF-Ab in 48 h. A) Left; Plot of the normalized fluorescence intensity up to 10 μm from the edge of each small cell-colony surrounded by patterned microbeads functionalized with VEGF-Ab (15 points represented from n=3 samples). Statistical significance; paired two-tailed t-test (*** p<0.001). Right; Brightfield and fluorescence microscopy images of negative control (NC, top) and small cell-colonies surrounded by microbeads (bottom) functionalized with VEGF-Ab after 48 h of secretion. B) Left; Plot of the normalized fluorescence intensity measured on blocks of 5 μm from the edge of the small cell-colony up to 40 μm away from it. Error bars correspond to the mean values±SD, 6 points analyzed from 3 samples. Right; Heat map of the fluorescence intensity observed in the edges of the small cell-colonies and up to 40 μm away from it.

FIG. 15. Direct detection of the secretion of VEGF by combined patterns of small cell-colonies and microbeads functionalized with VEGF-SSSA in 48 h. A) Left; Plot of the normalized fluorescence intensity up to 10 μm from the edge of each small cell-colony surrounded by patterned microbeads functionalized with VEGF-SSSA (15 points represented from n=3 samples). Statistical significance; paired two-tailed t-test (*** p<0.001). Right; Brightfield and fluorescence microscopy images of negative control (NC, top) and small cell-colonies surrounded by microbeads (bottom) functionalized with VEGF-SSSA after 48 h secreting. B) Left; Plot of the normalized fluorescence intensity measured on blocks of 5 μm from the edge of the small cell-colony up to 40 μm away from it. Error bars correspond to the mean values±SD, 5 points analyzed from 3 samples. Right; Heat map of the fluorescence intensity observed in the edges of the small cell-colonies and up to 40 μm away from it.

FIG. 16. Drawing of a PDMS stamp. Both the inlet (2 mm) and the outlet (1 mm) were punched. The bottom of the channel is enclosed with glass covers. The channel itself could be empty (top) or contain a series of pillars for the printing of protein spots (bottom).

FIG. 17. Schematic drawing of the PMMA culture wells.

FIG. 18. Schematic drawing of Printing and Vacuum Lithography. A) The PDMS micropillars can be wetted with a solution of adhesion proteins. B) After cleaning the PDMS slab, the channel is out into intimate contact with the glass substrate and degassed by vacuuming. C) Through the intimate contact of the micropillars with the substrate, cell adhesion proteins can be transferred replicating the bottom of the pillars. Through vacuuming of the PDMS slab, a microbeads suspension can be loaded and let flow through the channel, allowing the patterning of the microbeads around the protein dots.

FIG. 19. Effect of channel width on microbeads patterning. A) Plot of patterned microbeads density in the substrate (measured as microbeads per μm²) with increasing channel width (100, 200, 500 and 1000 μm). Error bars mean±SD (n=3 samples per experimental condition). B) Scanning electron microscope images of the patterned microbeads in the different scenarios.

FIG. 20. Effect of channel length on microbeads patterning. A) Plot of patterned microbeads density in the substrate (measured as microbeads per μm²) with increasing channel length (5, 10 and 20 mm). Error bars mean±SD (n=3 samples per experimental condition). B) Scanning electron microscope images of the patterned microbeads in the different scenarios.

FIG. 21. Effect of channel height on microbeads patterning. A) Plot of patterned microbead density in the substrate (measured as microbeads per μm²) with increasing channel height (13 and 25 μm). Error bars mean±SD (n=3 samples per experimental condition). B) Scanning electron microscope images of the patterned microbeads in the different scenarios.

FIG. 22. Effect of suspension concentration on microbeads patterning. A) Plot of patterned microbeads density in the substrate (measured as microbeads per μm²) with increasing streptavidin coated microbead suspension concentrations (3 10⁹, 6 10⁹, 3 10¹⁰, 6 10¹⁰, 1.5 10¹¹ and 3 10¹¹ microbeads mL⁻¹). Error bars mean±SD (n=3 samples per experimental condition). B) Scanning electron microscope images of the patterned microbeads in the different scenarios.

FIG. 23: Patterning of microbeads of different sizes. A) Brightfield and Atomic Force microscopy images of the 200 nm diameter microbeads patterns (left) and the 500 nm diameter microbeads patterns (right). B) Left; brightfield microscopy images of co-patterned hHF-MSCs with either 200 or 500 nm diameter microbeads functionalized with either 0% or 50% FGF-2 24 hours after cell adhesion. Right; graphical representation of FGF stimulation (number of cells at 24 h, normalized to the number of cells at 0 h) for all 4 patterning scenarios.

FIG. 24: Simultaneous direct FGF-2 stimulation and VEGF detection on patterned cell clusters. A; MSCs proliferation under different dosages of FGF-2: i) Plots of the normalized number of cells per array (normalized to time=0 h) for the cells co-patterned in 100 μm dots with 0% and 50% FGF-2 functionalized microbeads. Error bars mean±SD (n=3). ii) Bright field microscope images of the MSCs in arrays co-patterned with 0% or 50% FGF-2 functionalized microbeads. B; Direct detection of the secretion of VEGF by combined patterns of small cell-colonies and microbeads functionalized with anti-VEGF immunosandwich in 48 h: i) Plot of the normalized fluorescence intensity up to 10 μm from the edge of each small cell-colony surrounded by patterned microbeads (20 points represented from n=4 samples). Statistical significance; one-way ANOVA, post hoc Tukey. *** means statistical significance of p <0.001. ++ and +++means statistical significance of p<0.01 and p<0.001, respectively, compared to Control. ## #means statistical significance of p<0.001 compared to 0% FGF. ii) Bright field and fluorescence microscopy images of negative control and small cell-colonies surrounded by microbeads 0% and 50% FGF after 48 h of secretion.

### DETAILED DESCRIPTION OF THE INVENTION

The inventors of the present invention have developed a support that couples the controlled seeding of cells into predetermined patterns with the presentation and/or detection of molecules of interest to/from said cells. Said support presents several advantages related to previous ones such as precise control of the position and number of cells present in the predetermined patterns as well as presentation and or detection of molecules of interest due to the uniform covering of the support surface with microbeads functionalized with at least one molecule of interest.

### Support

Therefore, a first aspect of the present invention relates to a support suitable for culturing cells, from here onwards, “support of the invention”, characterized in that it shows patterned regions, said pattern being defined by a complementary pattern of uniformly distributed microbeads attached to the support and wherein the microbeads are modified with at least one functionalizing molecule, wherein the patterned regions in the support defined by the microbeads show increased adhesive capacity to cells with respect to the regions to which the microbeads are attached.

The expression “support” as used herein refers to a structure acting as a base which is able to support, sustain or maintain a cell or cells. The support as used herein can be made of any appropriate material such thermoplastics, thermosets, elastomers such as epoxy, phenolic, PDMS, glass, silicones, nylon, polyethylene, polystyrene, cyclic olefin copolymers or any other suitable material. In a preferred embodiment of the support of the invention, the support is made from a material selected from a group consisting of: glass, borosilicate glass, quartz glass, polystyrene, polymethylmethacrylate (PMMA), polycarbonate, polyethylene and cyclic olefin copolymers. In a more preferred embodiment the support is made of glass.

The expression “suitable for culturing cells” as used herein refers to any material in which cellular material is stored or maintained in a controlled environment so as to produce the conditions required for viability, i.e., the processes of cell culture. In a preferred embodiment the support is composed of a material which is hydrophilic, cytophilic and/or biocompatible. The term “hydrophilic” as used herein refers to a material which is attracted to or has an affinity to water. The term “cytophilic” as used herein refers to a material which is attracted to or has an affinity to cells. The term biocompatible as used herein refers to a material which has the ability to contact with a living system, such as cells, without producing adverse effects.

The term “cell” as used herein is to be understood as interchangeable with the term “cellular material” and shall refer to a cell, group of cells, tissue, or organoid which is the subject of the invention described herein.

The expression “microbeads attached to the support” as used herein refers to the fact that said microbeads are bound, joined, connected or in very close proximity to the uppermost plane of the support.

The term “microbeads” as used herein refers to micro-particles that may be spherical or oval or have an irregular shape. The microbeads can be organic, inorganic, synthetic, or natural materials. Examples of said materials are, without limitation, glass, polystyrene, poly (methyl methacrylate), silica, zirconia, titanium, gold, polyethylenepolylactic acid (PLA), poly-L-lactic acid (PLLA), poly glycolic acid (PGA), poly lactic-co-glycolic acid (PLGA) and poly-caprolactone (PCL). In a preferred embodiment of the support of the invention the microbeads are selected from a group consisting of: glass microbeads, polystyrene microbeads, polyacrylamide microbeads, poly (methyl methacrylate) microbeads, silica microbeads, zirconia microbeads, titanium microbeads, gold microbeads, polyethylenepolylactic acid (PLA) microbeads, poly-L-lactic acid (PLLA) microbeads, poly glycolic acid (PGA) microbeads, poly lactic-co-glycolic acid (PLGA) microbeads and poly-caprolactone (PCL) microbeads, preferably glass microbeads, polystyrene microbeads, poly (methyl methacrylate) microbeads, more preferably polystyrene microbeads.

In another preferred embodiment of the support of the invention the microbeads have a diameter of at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 2000 nm, at least 3000 nm, at least 4000 nm, at least 5000 nm.

In another preferred embodiment of the support of the invention the microbeads have a diameter of less than 25000 nm, less than 24000 nm, less than 23000 nm, less than 22000 nm, less than 21000 nm, less than 20000 nm, less than 19000 nm, less than 18000 nm, less than 17000 nm, less than 16000 nm, less than 15000 nm, less than 14000 nm, less than 13000 nm, less than 12000 nm, less than 11000 nm, less than 10000 nm, less than 10000 nm, less than 9000 nm, less than 8000 nm, less than 7000 nm, less than 6000 nm, less than 5500 nm.

In a preferred embodiment of the support of the invention the microbeads have a diameter of between 50 nm to 1000 nm, between 100 nm to 950 nm, between 150 nm to 900 nm, between 200 nm to 850 nm, between 250 nm to 800 nm, between 300 nm to 750 nm, between 350 nm to 700 nm, between 400 nm to 650 nm, between 450 nm to 600 nm, between 500 nm to 550 nm. In a preferred embodiment of the support of the invention the microbeads have a diameter of between 10000 nm to 25000 nm, between 11500 nm to 23500 nm, between 13000 nm to 22000 nm, between 14500 nm to 20500 nm, between 16000 nm to 19000 nm, between 16500 nm to 17500 nm, between 1000 nm to 10000 nm, between 2000 nm to 9000 nm, between 3000 nm to 8000 nm, between 4000 nm to 7000 nm, between 4000 nm to 6000 nm, between 4000 nm to 5000 nm.

In a preferred embodiment of the support of the invention the diameter of the microbeads is between 50 nm and 25000 nm.

The microbeads present on the surface of the support of the invention are “modified with at least one functionalizing molecule”. Said expression in the context of the present invention refers to the fact that the microbeads comprise a functional group or molecule bound to their surface wherein said functional group or molecule allows the detection and/or interaction of a specific molecule of interest in the medium or in the cell surface of cells that may be present in the support.

As used herein, the term “functionalizing molecule” refers to any molecule which can cause an effect on a cell (hereinafter known as “effector molecule”) or which can specifically bind to a molecule of interest (hereinafter known as “detecting molecule”). It will be understood that the chemical nature of the functionalizing molecule is not particularly limiting as long as the molecule can be attached to the microbeads and thus results in the functionalization of the microbead. In some embodiments, the functionalizing molecule is a small organic molecule, a peptide, a polypeptide, a nucleic acid, a carbohydrate or a lipid.

In a preferred embodiment, the functionalizing molecule is a high molecular weight compound.

As used herein, “high molecular weight compound” refers to any chemical entity or molecule, such as nucleic acids, peptides, proteins, natural and synthetic polymers, drugs, having a molecular weight greater than 1 kDa, preferably greater than 5 kDa, more preferably greater than 10 kDa and even more preferably greater than 100 kDa.

In one embodiment, the functionalizing molecule is a poplypeptide.

The term “polypeptide”, as used herein, refers to a polymer of amino acid residues. The term also apply to amino acid polymers in which one or more amino acid residue is an artificial chemical mimetic of a corresponding naturally occurring amino acid, as well as to naturally occurring amino acid polymers and non-naturally occurring amino acid polymer.

In one embodiment, the functionalizing molecule is a polynucleotide.

The terms “nucleic acid” and “polynucleotide”, as used herein interchangeably, refer to a polymer composed of nucleotide units (ribonucleotides, deoxyribonucleotides, related naturally occurring structural variants and synthetic non-naturally occurring analogs thereof or combinations thereof) linked via phosphodiester bonds, related naturally occurring structural variants and synthetic non-naturally occurring analogs thereof.

In one embodiment, the functionalizing molecule is a lipid.

The term “lipid” is used to indicate an organic compound that includes an ester of fatty acid or a derivative thereof and is characterized by being insoluble in water, but soluble in many organic solvents. Suitable lipids for use as functionalizing molecules include, without limitation, fatty acyls (FA), such as fatty acids, fatty acyl carnitines and fatty acyl Coenzyme As, glycerolipids (GL), such as monoacylglycerols, cardiolipins, diacylglycerols and triacylglicerols, glycerophospholipids (GPL) such as phosphatidylcholines, phosphatidylethanoloamines, and bis (monoacylglycerolphospahte (BMP), sphingolipids (SPL) such as ceramies, sphonglmyelins and cholesterylester, and sterol lipids (ST).

In another embodiment, the functionalizing molecule is a small organic molecule. In general, a “small molecule” refers to a substantially non-peptidic, non-oligomeric organic compound either prepared in the laboratory or found in nature. Small molecules, as used herein, can refer to compounds that are “natural product-like,” however, the term “small molecule” is not limited to “natural product-like” compounds. Rather, a small molecule is typically characterized in that it contains several carbon-carbon bonds, and has a molecular weight of less than 1500 g/mol, less than 1250 g/mol, less than 1000 g/mol, less than 750 g/mol, less than 500 g/mol, or less than 250 g/mol, although this characterization is not intended to be limiting for the purposes of the present invention. In certain other embodiments, natural-product-like small molecules are utilized.

Methods for the functionalization of the microbeads are well known in the art. In a preferred embodiment of the support of the invention the molecule is functionalized to the microbeads by Cu (I)-catalyzed azide-alkyne reaction, covalent attachment to amine groups present at the surface, surfactant addition or swelling. In another preferred embodiment of the support of the invention the molecule is functionalized through a coupling molecular pair, wherein one member of the coupling molecular pair is bound to the microbeads and the other member of the coupling molecular pair is bound to the molecule of interest. The term “coupling molecule pair” as used herein refers to two compounds which have a high affinity for each other producing stable and lasting interactions. Said coupling molecular pair can therefore be used to bind a molecule of interest to the surface of the microbeads. In said case, one of the members of the coupling molecule pair is functionalized to the microbead while the other member of the coupling molecule pair is bound to the molecule of interest. When in close proximity of each other the coupling molecular pair will bind together, effectively functionalizing the microbead with the molecule of interest. Examples of coupling molecular pairs are, without limitation, azide-alkyne, alkaline-nitrone and biotin-streptavidin. In a preferred embodiment of the support of the invention the coupling molecular pair is biotin-streptavidin molecular pair.

Functionalization of the microbeads with a molecule of interest can also be accomplished with one single coupling molecule which binds both the microbeads and the molecule of interest. Therefore, in a preferred embodiment of the support of the invention the molecule is functionalized through a single coupling molecule. The term “single molecule pair” as used herein refers to a compound which can bind together the surface microbeads and the molecule of interest, producing stable and lasting interactions. Examples of said single coupling molecule are, without limitation, low molecular weight heparin, polyethylene glycol (PEG), 1,4,7,10-tetraazacyclododecane-1,4,7,10-tetraacetic acid (DOTA), diethylene triamine pentaacetic acid (DTPA), silane coupling agents, like aldehyde-, amino-, and hydroxyl-silanes, for example 4-amino butyl triethoxysilane. In a preferred embodiment of the support of the invention the single coupling molecule is selected from a group consisting of: low molecular weight heparin, polyethylene glycol (PEG), 1,4,7,10-tetraazacyclododecane-1,4,7,10-tetraacetic acid (DOTA), diethylene triamine pentaacetic acid (DTPA), aldehyde-silanes, amino-silanes, hydroxyl-silanes or any combination thereof.

In a preferred embodiment of the support of the invention the microbeads attached to the support comprise microbeads with at least one functionalizing molecule and microbeads which are not functionalized, wherein the microbeads which are not functionalized may be functionalized while attached to the support. In another preferred embodiment of the support of the invention the microbeads attached to the support comprise at least two types of microbeads wherein each type of microbeads is modified with at least one functionalizing molecule and the functionalizing molecule is different between the different types of microbeads. In another preferred embodiment of the support of the invention the microbeads attached to the surface comprise at least two types of microbeads wherein each type of microbeads is modified with at least one functionalizing molecule, wherein the functionalizing molecules are distinct between the different types of microbeads and each type of microbeads is of and identical or different type of material or a combination thereof. In another preferred embodiment of the support of the invention the microbeads attached to the support comprise at least two types of microbeads wherein each type of microbead is modified with at least one functionalizing molecule, wherein the functionalizing molecules are identical or different between the different types of microbeads.

The expression “complementary pattern of uniformly distributed microbeads attached to the support” as used herein refers to the homogenous distribution of the microbeads within a predetermined pattern in the support surface. The microbeads can be considered as being uniformly distributed over the complementary pattern, if the complementary pattern is divided into areas of equal surface, the amount of microbeads in each area is substantially constant among the different areas. Thus, in some embodiments, the number of microbeads within two different areas varies by less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01% or even less. As the person skilled in the field will be aware, in order to obtain an uniform distribution of the microbeads in the support of the invention, the concentration/number of microbeads must be adjusted to the area of the support as well as the volume of the bottomless channels and suspension (see below) used to place the microbeads. This can be done by routine experimental techniques as exemplified in Example 3.

The term “predetermined patterned regions” as used herein refers to any type of free or geometric form which can be imprinted/established into the support material which determines the bounds of two regions which contain or do not contain microbeads. The term “patterned regions” does not imply that the form present in the support of the invention contains a motif which is repeated, as any free form or geometric form with or without a repeated motif is allowed. In a preferred embodiment of the support of the patterned regions are defined by a complementary pattern of uniformly distributed microbeads attached to the support and wherein the complementary pattern of uniformly distributed microbeads is created by allowing the microbeads to adhere to the support when the support is in contact with a stamp, said stamp having protrusions which define the pattern regions within the support, thereby preventing the microbeads from attaching to those areas of the support which are in contact with the stamp protrusions.

The terms “stamp” and “slab” are used interchangeably and in the present context refer to elastomeric stamps or slabs which are easily molded or formed into desired patterns or forms and used to allow the formation of the patterns of interest in the surface of the support. Materials suitable to be used as stamps are, without limitation, silicone rubbers (e.g., polydimethylsiloxane (PDMS)), polyurethane rubber, styrene butadiene rubber, and acrylonitrile butadiene rubber, natural rubbers (e.g., poly-cis-isoprene), thermoplastic elastomers (e.g., thermoplastic polyurethane, thermoplastic copolyester, thermoplastic polyamide), epoxies (e.g., SU-8), polyimides, polyurethanes, polyamides, polyesters (e.g., poly (lactic-co-glycolic acid) (PLGA), polylactic acid (PLA), polyglycolic acid (PGA), polycaprolactone (PCL), poly (glycerol sebacate) (PGS)), polysaccharides (e.g., chitosan), parylene, and combinations thereof.

The expression “complementary pattern” refers to the inverse shape/form of the pattern of interest such as the mold used to cast a metal tool has the inverse form of the tool.

As mentioned the stamp protrusions, when into contact with the support surface, prevent the microbeads to attach to those areas of the support. The term “protrusion” as used in the present description refers to projections areas of the stamp which are salient in respect to the average surface area of the stamp. These protrusions will eventually lead to “patterned regions in the support defined by the microbeads show increased adhesive capacity to cells with respect to the regions to which the microbeads are attached”. The term “region” as used herein refers to an area/local/part of the surface of the support which is somehow delimited from another region. In the present invention the attachment of microbeads to the support defines two delimited regions characterized in that one region has reduced adhesive capacity to cells, namely region which has the microbeads attached. In a preferred embodiment the patterned regions have an increased adhesive capacity to cells of at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2% in comparison to the regions with microbeads attached. In another preferred embodiment the patterned regions has an increased adhesive capacity to cells of at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20% in comparison to the regions with microbeads attached. In yet another preferred embodiment the patterned region has an increased adhesive capacity to cells of at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 100% in comparison to the regions with microbeads attached. The adhesive capacity of both regions can be determined by routine experimental procedures, such as, without limitation, seeding a cell solution in the support for a predetermined time, after which the solution is removed and the support is washed with a buffer solution. After washing, the number of cells attached to each region is counted and the reduction between regions proportional to area of said regions calculated.

In a preferred embodiment the regions in the support onto which the microbeads are attached are further coated with a compound or composition which substantially reduces cell adhesion. The expression “compound or composition which substantially reduces cell adhesion” in the present context refers to blocking agents which reduce the attachment of cells in the regions wherein they are present. Such blocking agents can be further be used in combination with solvents and/or buffers which allow their distribution through the region which is to have a reduced adhesive capacity to cells. Examples of such compounds or composition are, without limitation, bovine serum albumin (BSA), milk powder, polyetilenglycol (PEG), polyacrylamide, phosphatidylcholine, pluronic acids, dextran, self-assembly monolayers of closely packed amphiphilic molecules such as alkanethiols terminated with oligo (ethylene glycol) (OEG) or mixed OEG-OH and OEG-COOH, or alkanethiols terminated in positively charged moieties such as tri(methyl)ammonium (TMA) and negatively charged moieties such as the sulfate group (SO) and polymer brushes (Vaisocherová et al., Anal Bioanal Chem, 2015, 407:3927-3953).

The support comprises at least two types of regions, one of which has an increased adhesive capacity to cells in respect to the other region. The regions of the support which have microbeads attached to, are defined by the complementary pattern of the patterned regions, as previously defined. In a preferred embodiment of the support of the invention the patterned regions of the support which do not have microbeads attached to have a geometric form selected from a group consisting of: triangle, square, rectangle, rhombus, parallelogram, trapezoid, trapezium, n-agon, circle, ellipse or any combination thereof. In a more preferred embodiment of the support of the invention the patterned regions of the support have a geometrical form, preferably a circular form with the diameter of between 10 micrometers (μm) to 200 μm, preferably from 50 μm to 100 μm.

In a preferred embodiment of the support of the invention, the patterned regions in the support are coated with a compound or composition that promotes cell adhesion. In another preferred embodiment of the support of the invention, the coating of the support with a compound or composition that promotes cell adhesion is carried out by coating the protrusions of the stamp with said compound or composition that promotes cell adhesion and contacting the support with the stamp thereby allowing the transfer of the compound or composition from the stamp onto the support with a pattern that matches the pattern of protrusions of the stamp.

The expression “compound or composition which promotes cell adhesion” in the context of the present description refers to a protein located on the cell surface involved in the process of cell-cell or cell-extracellular matrix binding. The cell adhesion proteins includes immunoglobulins, integrins, cadherins and selectins. Non-limiting examples of compounds which promote cell adhesion are, without limitation, collagen types I, II and IV, elastin, fibronectin, vitronectin and laminin. In a preferred embodiment of the support of the invention the compound which promotes cell adhesion is an extracellular matrix molecule selected from the group which consists of: collagen type I, collagen type II, collagen type IV, elastin, fibronectin, vitronectin, laminin or any combination thereof, preferably fibronectin.

The term “extracellular matrix” or its acronym “ECM” as used herein refers to the non-cellular component present within all tissues and organs, which provides not only essential physical scaffolding for the cellular constituents but also initiates crucial biochemical and biomechanical cues that are required for tissue morphogenesis, differentiation and homeostasis.

In the context of the present invention the expression “allowing the transfer of the compound or composition from the stamp onto the support with a pattern that matches the pattern of protrusions of the stamp” refers to process of binding, joining or connecting said compound or composition to the surface of the support in such a way as to form a pattern in the support which will determine the regions within the support of the invention onto which the cells will adhere. Said process can be done by a variety of methods which the skilled person in the art will be knowledgeable about, collectively known as soft-lithography, such as micro-contact printing, micro-molding in capillary, replica molding, solvent-assisted micro-molding, phase-shifting edge lithography, decal transfer lithography, nanotransfer printing, dip-pen lithography, nano-skiving and vacuum-drive soft lithography. In a preferred embodiment of the support of the invention the patterned regions are created by imprinting the support with stamps containing the compound or composition which promotes cell adhesion using a technique selected from the group consisting of: micro-contact printing, micro-molding in capillary, replica molding, solvent-assisted micro-molding, phase-shifting edge lithography, decal transfer lithography, nanotransfer printing, dip-pen lithography, nano-skiving and vacuum-driven soft lithography, preferably microcontact printing, preferably vacuum-driven soft lithography. In another preferred embodiment of the support of the invention the patterned regions are created using vacuum-driven soft lithography.

As used herein, the term “microcontact printing” is meant to designate any process useful for applying a molecule onto a surface, preferably such that the dimensions of the printed structures lie in the μm-range and/or in the nm-range. In a preferred embodiment of the support of the invention the patterned regions are created by microcontact printing with the use of stamps comprising a elastomer selected from a group consisting of: PDMS, PDMA, polyurethane rubber, styrene butadiene rubber, acrylonitrile butadiene rubber, poly-cis-isoprene, thermoplastic polyurethane, thermoplastic copolyester, thermoplastic polyamide, SU-8, polyimides, polyurethanes, polyamides, PLGA, PLA, PGA, PCL, PGS, chitosan, parylene or any combination thereof, preferably PDMS.

In a preferred embodiment of the support of the invention the support further contains viable cells which are attached to the support or to the molecules within the support which promotes cell adhesion. The term “viable cells” as used herein refers to cells which are capable of proliferate in a suitable medium as well as cells which are capable of maintaining a basal metabolism suitable for their functions without dividing, indicative of a healthy cell state. In a preferred embodiment of the support of the invention the support further contains cells, wherein the cells are insect cells, avian cells, mammalian cells, hybridoma cells, primary cells, continuous cell lines, stem cells and/or genetically engineered cells, and wherein the genetically engineered cells are recombinant cells expressing a heterologous protein or polypeptide. In another preferred embodiment of the support of the invention, the support further contains animal cells, wherein the animal cells are mammalian cells, preferably selected from a group consisting of: BSC-1 cells, LLC-MK cells, CV-1 cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO. perC6 (human retina cells) or derivatives thereof. In another preferred embodiment of the support of the invention, the support further contains insect cells, wherein the insect cells are selected from a group consisting of: Sf21, Sf9, and the BTI-TN-5B1-4 (or High Five) cells, or any combination thereof.

The term “avian cells” as used herein refers to cells or cell cultures with cells originated from birds. A list of acceptable lines which can be used in the context of the present invention is available in the literature (K. Nazerian, 1987, Avian Pathology, 16:3, 527-544).

The term “insect cells” as used herein refers to cells originated from insects, preferably lepidopteran cell lines. A list of available cell line from lepidopteran is available (Lynn D., 2007, pp. 118-129, In: Murhammer D. W. (Eds) Baculovirus and Insect Cell Expression Protocols. Methods in Molecular BiologyTM, vol 388. Humana Press. In another preferred embodiment of the support of the invention the cells are insect cells, wherein the insect cells are selected from a group consisting of: Sf21, Sf9, and the BTI-TN-5B1-4 (or High Five) cells, or any combination thereof.

The term “hybridoma cell” as used herein refers to a hybrid cell obtained from the fusion of an antibody producing B-cell of interest and an immortal B cell cancer cell, a myeloma. These cells allow the production of antibodies of interest, monoclonal antibodies.

The term “primary cell” in the context of the present description refers to cells freshly obtained from a multicellular organism which are culture ex vivo.

The term “stem cell” as used herein refers to an undifferentiated cell that has the ability to divide and replicate itself for an indefinite period while maintaining an undifferentiated state even ex vivo. Furthermore, the stem cell has the ability to differentiate into many kinds of different cells in an organism depending on the developmental stage and location of an individual. Stem cells can be totipotent, pluripotent or multipotent.

In another preferred embodiment of the support of the invention the support further contains stem cells, preferably induced stem cells, more preferably mesenchymal stem cells (MSC). The term “mesenchymal stem cells” or its acronym “MSC” as used herein refers to mesenchymal and/or adipose stem cells. In a preferred embodiment of the support of the invention the support comprises MSC cells selected from a group consisting of: bone-marrow MSCs, Adipose MSCs, Umbilical Cord MSCs, ES Cell Derived MSCs and hair follicle derived MSCs, preferably hair follicle derived MSCs.

### Method for the Production of the Support of the Invention

Another aspect of the present invention relates to a method for producing the support of the invention, from here onwards the producing method of the invention, comprising the steps of:
(i) providing a support;
(ii) contacting the support with a stamp, said stamp having protrusions which define the desired pattern in the support, wherein the contact of the support with the stamp forms bottomless channels between the support and the stamp and
(iii) contacting the support along the bottomless channels with a microbead suspension and allowing the microbeads to adhere to the support as to obtain a uniform deposition and attachment of the microbeads onto those regions in the support which are not protected by the protrusions of the stamp.

All the previous defined terms and expressions are equally valid for the present aspect and its embodiments.

In a first step, a support is provided. In some embodiments, the support of step (i) is made from a material selected from a group consisting of: glass, borosilicate glass, quartz glass, polystyrene, polymethylmethacrylate (PMMA), polycarbonate and polyethylene, preferably glass.

In a second step, the method according to the present invention involves contacting the support with a stamp, said stamp having protrusions which define a desired pattern, wherein the contact of the support with the stamp forms bottomless channels between the support and the stamp.

The expression “contact of the support with the stamp” as used herein refers to the process of bringing the support and the stamp into very close contact thereby allowing the protrusions of the stamp to touch the surface of the support. This sandwich type process, wherein the support and the stamp are place onto each other prevents the microbeads from attaching to the support surface in those regions where the protrusions touch the surface of the support.

In a preferred embodiment of the producing method of the invention the areas of the support which are in contact with the stamp in step (ii) are coated with a compound or composition that promotes cell adhesion. In another preferred embodiment of the producing method of the invention the protrusions of the stamp in step (ii) are coated with a compound or composition that promotes cell adhesion thereby allowing the transfer of the compound or composition from the stamp onto the support with a pattern that matches the pattern of protrusions of the stamp.

In a preferred embodiment of the producing method of the invention the stamp of step (ii) is made of an elastomer selected from a group consisting of: PDMS, polyurethane rubber, styrene butadiene rubber, acrylonitrile butadiene rubber, poly-cis-isoprene, thermoplastic polyurethane, thermoplastic copolyester, thermoplastic polyamide, SU-8, polyimides, polyurethanes, polyamides, PLGA, PLA, PGA, PCL, PGS, chitosan, parylene or any combination thereof, preferably PDMS.

In a preferred embodiment, step (ii) is carried out by vacuum-driven soft lithography. In some embodiments, step (ii) is carried out by applying pressure onto the stamp or onto the support or onto both the stamp and the support. In some embodiments, the pressure is achieved by creating vacuum in the chamber formed between the support and the stamp. When the chamber is provided with operable inlet and an operable outlet, both outlets are closed so as to allow the generation of vacuum in the chamber.

In a preferred embodiment of the producing method of the invention the patterned regions of the support have a geometric form selected from a group consisting of: triangle, square, rectangle, rhombus, parallelogram, trapezoid, trapezium, n-agon, circle, ellipse or any combination thereof. In a more preferred embodiment of the support of the invention the patterned regions are circles with the diameter between 10 micrometers (μm) to 200 μm, preferably from 50 μm to 100 μm.

In the present producing method of the invention the attachment of microbeads to the support defines two delimited regions, the patterned regions and the complementary regions, characterized in that the latter region has reduced adhesive capacity to cells. In a preferred embodiment the complementary region with microbeads attached has a reduced adhesive capacity to cells of more than at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2% less than the patterned regions. In another preferred embodiment the complementary regions has a reduced adhesive capacity to cells of more than at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20% less than the patterned regions. In yet another preferred embodiment the complementary region has a reduced adhesive capacity to cells of more than at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 100% than the patterned regions.

In a preferred embodiment of the producing method of the invention the compound or composition which promotes cell adhesion of step (ii) is an extracellular matrix molecule selected from the group which consists of: collagen type I, collagen type II, collagen type IV, elastin, fibronectin, vitronectin, laminin or any combination thereof, preferably fibronectin.

In some embodiments, the stamp and the support are placed so that a chamber is formed between these two elements, said chamber being provided with an operable inlet and an operable outlet.

Step (iii) of the method for the production of the support of the invention refers to the deposition of microbeads in complementary region onto the surface of the support, wherein said deposition is made by allowing the beads to flow along the bottomless channels formed in between the stamp and the support, wherein said bottomless channels are present due to the fact that the protrusions present in the stamp which form contact zones in step (ii) of the producing method of the invention are elevated in height in relation to the channels. Step (iii) allows the uniform or homogeneous deposition of microbeads in a complementary pattern onto the surface of the support. The term “bottomless channels” as used herein refers to the fact the said channels are opened at both extremes, therefore, lacking a base or bottom.

In some embodiments of the producing method of the invention, in step (iii) the chamber formed between the stamp and the support is provided with an operable inlet and an operable outlet and said step (iii) is carried out by injecting a suspension of microbeads through the inlet while the outlet is closed, maintaining the suspension within the channels for a sufficient time to allow uniform deposition of the microbeads on the support and extracting the suspension through the outlet. In some embodiments step (iii) is carried out while pressure is being applied to bring into contact the slab and the support. In some embodiments the pressure is achieved by creating vacuum in the chamber formed between the support and the slab. In some embodiments, the suspension containing microbeads is placed in the inlet while the outlet is sealed allowing the suspension to enter the channels due to the negative pressure therein as a result of the vacuum applied in step (ii). In some embodiment the suspension containing microbeads is allowed to flow for 1 min to 10 minutes, preferably 2 min to 8 min, more preferably for 5 min. In some embodiment the suspension containing microbeads is allowed to flow until the outlet is filled. In some embodiment the outlet is unsealed after the suspension containing microbeads has filled the outlet and the suspension is allowed to flow out of the outlet.

In a preferred embodiment of the producing method of the invention the suspension of microbeads injected in step (iii) further contains a compound or composition which substantially reduces cell adhesion. In another preferred embodiment of the producing method of the invention the regions in the support onto which the microbeads are attached in step (iii) are further coated with a compound or composition which substantially reduces cell adhesion. Examples of said compounds or compositions were provided above and said examples are equally valid for the present method of the invention.

In a preferred embodiment of the producing method of the invention the microbeads of step (iii) are selected from a group consisting of: glass microbeads, polystyrene microbeads, polyacrylamide microbeads, poly (methyl methacrylate) microbeads, silica microbeads, zirconia microbeads, titanium microbeads, gold microbeads, polyethylenepolylactic acid (PLA) microbeads, poly-L-lactic acid (PLLA) microbeads, poly glycolic acid (PGA) microbeads, poly lactic-co-glycolic acid (PLGA) microbeads and poly-caprolactone (PCL) microbeads, preferably glass microbeads, polystyrene microbeads, poly (methyl methacrylate) microbeads, more preferably polystyrene microbeads.

In a preferred embodiment of the producing method of the invention the microbeads of step (iii) have a diameter between 50 nm and 25000 nm.

In some embodiments, after the microbeads containing the functionalizing molecule are deposited on the support, the support is then contacted with a quenching agent that reduces non-specific binding of any other molecule that is contacted with the support. Suitable quenching agents that can be used include, without limitation, milk powder, bovine serum albumin, heat-inactivated serum or a combination of one or more of the above.

In a preferred embodiment of the producing method of the invention the solution of microbeads of step (iii) comprises microbeads with at least one functionalizing molecule and microbeads which are not functionalized, wherein the microbeads which are not functionalized may be functionalized while attached to the surface of the support.

The preferred microbeads sizes used for the producing method of the invention have been previously defined for the support of the invention and said sizes are equally valid for the present aspect.

In some embodiments, steps (ii) and (iii) are carried simultaneously, which means that the microbeads and the compound which promotes cell adhesion are contacted with the support at the same time.

In some embodiments, the producing method of the invention further comprises contacting the support with a cell population under conditions adequate for the binding of the cells to the support through interactions between the cells and the compound in the support that promotes cell adhesion and the formation of patterned cell clusters.

The term “cell population” as used herein is synonymous with the term “cell culture”. In a preferred embodiment of the producing method of the invention the cell population contacted with the support is selected from a group consisting of: insect cells, avian cells, mammalian cells, hybridoma cells, primary cells, continuous cell lines, stem cells and/or genetically engineered cells, and wherein the genetically engineered cells are recombinant cells expressing a heterologous protein or polypeptide.

In another preferred embodiment of the producing method of the invention the cell population contacted with the support is an animal cell population, wherein the animal cells are mammalian cells, preferably selected from a group consisting of: BSC-1 cells, LLC-MK cells, CV-1 cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO. perC6 (human retina cells) or derivatives thereof.

In another preferred embodiment of the producing method of the invention the cell population contacted with the support is an insect cell population, wherein the insect cells are selected from a group consisting of: Sf21, Sf9, and the BTI-TN-5B1-4 (or High Five) cells, or any combination thereof.

In another preferred embodiment of the effect method of the invention the cell population contacted with the support is a stem cell population, preferably mesenchymal stem cells (MSC), wherein the MSC cells are selected from a group consisting of: bone-marrow MSCs, Adipose MSCs, Umbilical Cord MSCs, ES Cell Derived MSCs and hair follicle derived MSCs, preferably hair follicle derived MSCs.

In order to contact the support with a cell population a structure which maintains the cell population as well as its adequate medium may be required. In a preferred embodiment of the producing method of the invention the cell population contacting the support is placed inside a well-like structure wherein said well-like structure is formed by a base and a vertical wall or walls, wherein the base is formed by the support layer comprising the predetermined patterns base.

The term “well-like structure” in the present context refers to an enclosure or compartment formed by a base, a vertical wall or walls and an open top. Said base is the support which contains the patterned regions onto which the vertical wall or walls are placed or in which the wall or walls are assembled, creating a seal between the base and the vertical wall or walls, said seal allowing to retain liquids or solutions which are required for the viability of the cell population placed in contacted with the support. The term “well-like structure” does not imply any shape or form of said structure, as the functional effect required by the structure can be achieved by any shape or form of the walls, such as one single circular wall or four walls forming a square. Furthermore, the well-like structure vertical walls can be preassembled and placed onto the support, such as, without limitation, bottomless wells formed by PDMS/PDMA slabs which are placed onto the support, or be assembled directly on the support, such as, without limitation, hydrogels or silicone which can be placed onto the support in liquid form and allowed to solidify to form the well-like structure. In addition, as the skilled person in the field will be aware, materials may be required to seal the well-like structure, and said materials are well known in the field, with the only consideration of use being an inert material in relation to the cell population. Furthermore, the well-like structure may be treated agents in order to saturate and/or block protein-binding sites on the walls as to prevent unwanted interactions between the cells and the structure. Said agents are well known by the skilled person in the field and part of routine experimentation work. Examples include, without limitation, bovine serum albumin (BSA) and powder milk.

In a preferred embodiment of the producing method of the invention the cell population is placed in contact with the support inside a well-like structure wherein said well-like structure comprises vertical walls made from a material selected from a group consisting of: polydimethylsiloxane (PDMS), glass, poly (methyl methacrylate) (PMMA), pressure sensitive adhesive sheets (PSA), cyclic olefin copolymer (COC), cyclic olefin polymers (COP), polyethylene terephthalate (PET), BioMed Clear Resin, polyether ether ketone (PEEK), polystyrene (PS), polypropylene (PP), polyethylene (PE), polycarbonate or any combination thereof.

The term BioMed Clear Resin refers to the commercially available hard resin from FormLabs (https://formlabs-media.formlabs.com/datasheets/2001432-TDS-ES-0.pdf])

### Method for Determining the Effect of a Molecule in the Cell Culture

Another aspect of the present invention relates to a method for determining the effect of the functionalizing molecule on a cell population, from here onwards the effect method of the invention, which comprises the steps of:
(i) Providing a support according to the invention wherein said support contains the cell population wherein the cells of the population are attached to the support and wherein the molecule, the effect of which on the cell population is to be determined, is the functionalizing molecule;
(ii) Maintaining the support under adequate conditions to allow the functionalizing molecule to exert its effect on the cells of the cell population; and
(iii) Detecting the response of the cells within the cell population to said functionalizing molecule,
   wherein the detection of a response of the cells to the functionalizing molecule is indicative that the functionalizing molecule exerts an effect on the cells of the cell population.

All the previous defined terms and expressions are equally valid for the present aspect and its embodiments.

In a first step, the method for determining the effect of a molecule in the cell culture comprises providing a support according to the invention wherein said support contains the cell population wherein the cells of the population are attached to the support by adhesion to the compound promoting cell adhesion and wherein the functionalizing molecule is the molecule of which the effect on the cell population is to be determined.

In a preferred embodiment of the effect method of the invention the cell population in step (i) is selected from a group consisting of: insect cells, avian cells, mammalian cells, hybridoma cells, primary cells, continuous cell lines, stem cells and/or genetically engineered cells, and wherein the genetically engineered cells are recombinant cells expressing a heterologous protein or polypeptide.

In another preferred embodiment of the effect method of the invention the cell population in step (i) is an animal cell population, wherein the animal cells are mammalian cells, preferably selected from a group consisting of: BSC-1 cells, LLC-MK cells, CV-1 cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO. perC6 (human retina cells) or derivatives thereof.

In another preferred embodiment of the effect method of the invention the cell population is step (i) is an insect cell population, wherein the insect cells are selected from a group consisting of: Sf21, Sf9, and the BTI-TN-5B1-4 (or High Five) cells, or any combination thereof.

In another preferred embodiment of the effect method of the invention the cell population is step (i) is a stem cell population, preferably mesenchymal stem cells (MSC), wherein the MSC cells are selected from a group consisting of: bone-marrow MSCs, Adipose MSCs, Umbilical Cord MSCs, ES Cell Derived MSCs and hair follicle derived MSCs, preferably hair follicle derived MSCs.

In a preferred embodiment of the effect method of the invention the compound which promotes cell adhesion of step (i) is an extracellular matrix molecule selected from the group which consists of: collagen type I, collagen type II, collagen type IV, elastin, fibronectin, vitronectin, laminin or any combination thereof, preferably fibronectin.

In another preferred embodiment of the effect method of the invention the functionalizing molecule of step (i) is selected from a group consisting of: growth factors, differentiation factors, cell adhesion molecules or proteins, cytokines, pharmaceutical small molecules and any combination thereof.

The term “growth factor” as used herein refers to any molecule or protein that specifically stimulates target cells to proliferate, differentiate, or alter their function or phenotype. In another preferred embodiment of the effect method of the invention the functionalizing molecule of step (i) is a growth factor selected from the group consisting of: vascular endothelial growth factor (VEGF), collagen, bone morphogenic factor-β, epidermal cell growth factor (EGF), platelet derived growth factor (PDGF), nerve growth factor (NGF), fibroblast growth factor (FGF), insulin-like growth factor (IGF), transforming growth factor (TGF) and any combination thereof.

The term differentiation factor as used herein refers to a molecule that promotes the differentiation of cells. In another preferred embodiment of the effect method of the invention the molecule found functionalizing the microbeads of step (i) is a differentiation factor selected from the group consisting of: neurotrophin, colony stimulating factors (CSF), transforming growth factor (TGF) and any combination thereof.

As used herein, the term “adhesive molecule or protein” or “cell adhesion molecule or protein” refers to a molecule or protein that promotes attachment of a cell to a bead and/or fibril. In preferred embodiment of the effect method of the invention the molecule found functionalizing the microbeads of step (i) is an adhesion molecule protein selected from the group consisting of: integrins, cadherins, selectins and any combination thereof.

The term “cytokine” is a generic term for proteins released by one cell population that act on another cell population as intercellular mediators. Examples of such cytokines are lymphokines, monokines, and traditional polypeptide hormones. Included among the cytokines are growth hormone include, such as human growth hormone, N-methionyl human growth hormone, and bovine growth hormone; parathyroid hormone; thyroxine; insulin; proinsulin, relaxin, prorelaxin, glycoprotein, hormones such as follicle stimulating hormone (FSH), thyroid stimulating hormone (TSH), and luteinizing hormone (LH), hepatocyte growth factor, fibroblast growth factor, prolactin, placental lactogen, factor-a and tumor growth-β; Mullerian inhibiting substance, gonadotropin-associated peptide mouse, inhibin, activin, vascular endothelial growth factor, integrin, thrombopoietin (TPO), nervous factors, such as EGF-β growth, platelet growth factor, transforming growth factors (TGF) such as TGF-α and TGF-β, insulin-like factor-I and -II growth, Erythropoietin (EPO), osteoinductive factors, interferons, such as interferon-a, interferon-β, and interferon-γ, colony stimulating factors (CSF) such as macrophage-CSF (M-CSF), CSF granulocyte-macrophage (GM-CSF), and granulocyte-CSF (G-CSF), interleukins (ILs) such as IL-1, IL-la, IL-2, IL-3, IL-4, IL-5, IL-6, IL-7, IL-8, IL-9, IL-11, IL-12; tumor necrosis factor, TNF-α as TNF-β; and other polypeptide factors including LIF and kit ligand (KL). As used herein, the term cytokine includes proteins from natural sources or from recombinant cell culture and biologically active equivalents of the native sequence cytokines. In a preferred embodiment of the effect method of the invention the molecule found functionalizing the microbeads of step (i) is selected from a group consisting of: human growth hormone, N-methionyl human growth hormone, bovine growth hormone, parathyroid hormone, thyroxine, insulin, proinsulin, relaxin, prorelaxin, glycoprotein, follicle stimulating hormone (FSH), thyroid stimulating hormone (TSH), luteinizing hormone (LH), hepatocyte growth factor, fibroblast growth factor, prolactin, placental lactogen, factor-a, tumor growth-β, Mullerian inhibiting substance, gonadotropin-associated peptide mouse, inhibin, activing, vascular endothelial growth factor, integrin, thrombopoietin (TPO), EGF-β growth, platelet growth factor, transforming growth factors (TGF)-α and TGF-β, insulin-like factor-I and -II growth, Erythropoietin (EPO), osteoinductive factors, interferon-α, interferon-β, interferon-γ, macrophage-CSF (M-CSF), CSF granulocyte-macrophage (GM-CSF), granulocyte-CSF (G-CSF) interleukin (IL)-1, IL-la, IL-2, IL-3, IL-4, IL-5, IL-6, IL-7, IL-8, IL-9, IL-11, IL-12; tumor necrosis factor (TNF)-α, TNF-β, Leukemia Inhibitory Factor (LIF) and kit ligand (KL).

The term “pharmaceutical small molecules” as used herein refers to molecules with a low molecular weight, preferably bellow 8,000 Daltons which can affect other molecules in the surface of the cells such as proteins and lipids leading to a response of the cells, normally cell death. Several databases of small molecules are available such as ChEML (https://www.ebi.ac.uk/chemb), DrugBank (https://go.drugbank.com) and PubChem (https://pubchem.ncbi.nlm.nih.gov) amongst others.

In a second step, the method for determining the effect of a molecule in the cell culture comprises maintaining the support under adequate conditions to allow the functionalizing molecule to exert its effect on the cells of the cell population. Said step further comprises maintaining the support under conditions in which the cells are maintained in a viable status so that any response to the functionalizing molecule is preserved. Typically, this step requires culturing the cells in the adequate conditions to detect the effect of the molecule of interest in the cell culture. This refers to the process of maintaining the cell population viability by supplying the cells with adequate culture media as well as support media additives and placing the cell population under the correct temperature, humidity, light and agitation conditions for the cells to remain viable. Examples of culture media and support media additives are, without limitation, Dulbecco's modified eagle's medium (DMEM), modified eagle's medium (MEM), eagle's basal medium (BME), Roosevelt Park Memorial Institute medium (RPMI), F12 medium, phosphate buffered saline (PBS), L-glutamine, L-alanyl-Lglutamate, non-essential amino acids (NEAA), fetal bovine serum (FBS), bovine calf serum (BCS), horse serum (HS), bovine serum albumin (BSA), human serum albumin (HSA), sodium bicarbonate, sodium carbonate, sodium pyruvate, lipoic acid, ascorbic acid, vitamin B12, nucleosides, cholesterol, oxygenating factors (perfluorocarbons (PFCs), sodium percarbonate, calcium peroxide, magnesium peroxide, hydrogen peroxide), apo-transferrin, insulin, reducing factors (glutathione), Wharton's jelly, and transferrin.

The correct conditions for each cell population is part of the general knowledge of the skilled person in the field.

In a third step, the method for determining the effect of the functionalizing molecule on a cell population comprises detecting the response of the cells within the cell population to said functionalizing molecule.

The expression “Detecting the response of the cells within the cell population to said molecule of interest” as used herein refers to the process of identifying at least one parameter that changes in relation to a cell population which is not in contact with the molecule of interest. Said change may be an increase or decrease in the value of said parameter. Said parameter may be a physical parameter of cells, such as cell number, cell proliferation, cell spreading, cell adhesion, cell migration, cell viability, cell secretion, cell growth, cell differentiation, cell morphology, cell apoptosis or any combination thereof. The measurement of cell number is routine experimentation practice and can be accomplished by counting the number of cells in brightfield images acquired just after cell deposition into the support and after one or more determined set times of interest. Cell proliferation can be easily measured by methods and techniques described elsewhere (Chung et al., 2017, Cytometry A. Jul; 91 (7): 704-712; Präbst et al., 2017, Methods Mol Biol. 1601:1-17; Romar et al., 2016, J Invest Dermatol. 2016 January; 136 (1): e1-e7). Cell spreading measurement methods are also described elsewhere (Ersoy et al., 2008, ed Image Comput Comput Assist Interv. 11 (Pt 1): 376-83) as are methods to measure cell proliferation, cell adhesion, cell spreading and cell apoptosis (Minor et al., Comb Chem High Throughput Screen 2008 August; 11 (7): 573-80). Techniques to measure cell growth, proliferation and apoptosis are also common knowledge of the skilled person in the field (Butler et al., 2014, Methods Mol Biol. 1104:169-92). Cell migration can be determined by time lapse microscopy wherein a sequential series of images of the cell population is acquired allowing the movement of cells to be determined and their instantaneous displacement as well as their overall displacement measured. Cell differentiation measurements will depend on the type of differentiation process occurring. Despite this the skilled person in the field will be aware of the best method to determine the differentiation level. Most methods rely on the detection of specific markers which indicate differentiation of a stem cell into a specific cell type or lineage. Just detection can be accomplished by labelling said markers with antibodies or with labels through genetic engineering, wherein said labels can in turn be detected by other methods, such as fluorescence microscopy, western blot, enzymatic assays, etc. Cell morphology and cell secretion can be determined and measured by techniques describe in the literature (Lamers et al., 2010, ur Cell Mater. November 9; 20:329-43; Torres et al., 2014, Anal. Chem. 86, 23, 11562-11569).

Furthermore, the cell response may be detected by the appearance, disappearance, reduction or increase of a label presented in said cells, wherein said label may be detected in the cell cytosol, surface or in the culture medium. Said label may be a natural label, such as cytokines, or artificial labels such as fluorescent proteins. In another preferred embodiment of the effect method of the invention the response of the cells detected in step (iii) is selected from a group consisting of: cell number, cell proliferation, cell spreading, cell attachment, cell adhesion, cell migration, cell viability, cell secretion, cell growth, cell differentiation, cell morphology, cell death, or any combination thereof.

### Method for Determining the Capacity of a Cell Population to Secrete a Substance of Interest

Another aspect of the present invention relates to a method for determining if a cell population secretes to the medium a substance of interest, which comprises the steps of:
(i) Providing a support according to the invention wherein said support contains the cells population wherein the cells of the population are attached to the support and wherein the functionalizing molecule comprises a molecule which is capable of interacting with the substance of interest, said interaction resulting in a detectable signal,
(ii) Culturing the cells in the adequate conditions to allow binding of the substances of interest secreted to the medium to the functionalizing molecule; and
(iii) Detecting the binding of the substance of interest to the molecule of interest by detecting the signal resulting from said interaction.

All the previous defined terms and expressions are equally valid for the present aspect and its embodiments.

In a first step, the method for determining the capacity of a cell population to secrete a substance of interest comprises providing a support according to the invention wherein said support contains the cell population wherein the cells of the population are attached to the support by adhesion to the compound promoting cell adhesion and wherein the functionalizing molecule comprises a molecule which is capable of interacting with the substance of interest, said interaction resulting in a detectable signal.

It will be understood that the method of the invention is useful not only for the detection of substances which are secreted by the cells and released into the surrounding medium but also for the detection of cellular components which are exposed to the outside of the cell but still associated with the cell such as, for instance, a membrane protein which, upon expression, is targeted to the membrane wherein the luminal domain is exposed to the outside of the cell, becoming available for binding to a specific ligand which is coupled to the microbead as functionalizing molecule.

In a preferred embodiment of the secretion method of the invention the cell population in step (i) is selected from a group consisting of: insect cells, avian cells, mammalian cells, hybridoma cells, primary cells, continuous cell lines, stem cells and/or genetically engineered cells, and wherein the genetically engineered cells are recombinant cells expressing a heterologous protein or polypeptide.

In another preferred embodiment of the secretion method of the invention the cell population in step (i) is an animal cell population, wherein the animal cells are mammalian cells, preferably selected from a group consisting of: BSC-1 cells, LLC-MK cells, CV-1 cells, CHO cells, COS cells, murine cells, human cells, Hela cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO. perC6 (human retina cells) or derivatives thereof.

In another preferred embodiment of the secretion method of the invention the cell population is step (i) is an insect cell population, wherein the insect cells are selected from a group consisting of: Sf21, Sf9, and the BTI-TN-5B1-4 (or High Five) cells, or any combination thereof.

In another preferred embodiment of the secretion method of the invention the cell population is step (i) is a stem cell population, preferably mesenchymal stem cells (MSC), wherein the MSC cells are selected from a group consisting of: bone-marrow MSCs, Adipose MSCs, Umbilical Cord MSCs, ES Cell Derived MSCs and hair follicle derived MSCs, preferably hair follicle derived MSCs.

In a preferred embodiment of the secretion method of the invention the compound which promotes cell adhesion of step (i) is an extracellular matrix molecule selected from the group which consists of: collagen type I, collagen type II, collagen type IV, elastin, fibronectin, vitronectin, laminin or any combination thereof, preferably fibronectin.

The term “substance of interest” as used herein refers to any substance, molecule or protein which the cell population secretes or displays to the culture medium and which is susceptible of being detected by the molecule functionalized to the microbeads. Examples of a substance of interest are biological nanoparticles such as extracellular vesicles or exosomes, cytokines, ions, heavy metal ions, carbohydrates, gases, reactive species, pH and any combination thereof. In a preferred embodiment of the secretion method of the invention the substance of interest of step (i) is selected from a group consisting of: exosomes, extracellular vesicles, lipoproteins, ferritin, viruses, human growth hormone, N-methionyl human growth hormone, bovine growth hormone, parathyroid hormone, thyroxine, insulin, proinsulin, relaxin, prorelaxin, glycoprotein, follicle stimulating hormone (FSH), thyroid stimulating hormone (TSH), luteinizing hormone (LH), hepatocyte growth factor, fibroblast growth factor, prolactin, placental lactogen, factor-a, tumor growth-β, Mullerian inhibiting substance, gonadotropin-associated peptide mouse, inhibin, activing, vascular endothelial growth factor, integrin, thrombopoietin (TPO), EGF-β growth, platelet growth factor, transforming growth factors (TGF)-α and TGF-β, insulin-like factor-I and -II growth, Erythropoietin (EPO), osteoinductive factors, interferon-α, interferon-β, interferon-γ, macrophage-CSF (M-CSF), CSF granulocyte-macrophage (GM-CSF), granulocyte-CSF (G-CSF) interleukin (IL)-1, IL-la, IL-2, IL-3, IL-4, IL-5, IL-6, IL-7, IL-8, IL-9, IL-11, IL-12; tumor necrosis factor (TNF)-α, TNF-β, Leukemia Inhibitory Factor (LIF), kit ligand (KL), glucose, lactate, oxygen (O₂), calcium (Ca²⁺), carbon dioxide (CO₂), Hydrogen peroxide (H₂O₂), sodium (Na⁺), potassium (K⁺), cadmium (Cd²⁺), cobalt (Co²⁺), palladium (Pb²⁺), copper (Cu²⁺), silver (Ag), magnesium (Mg²⁺), manganese (Mn²⁺), nickel (Ni²⁺), zinc (Zn²⁺), chromium (Cr³⁺), iron (Fe³⁺), aluminum (Al³⁺), mercury (Hg²⁺) and any combination thereof.

The expression “the functionalizing molecule comprises a molecule which is capable of interacting with the substance of interest” refers to the process of the functionalizing molecule interacting by binding, processing or reacting with the substance or interest.

In a second step, the method for determining the capacity of a cell population to secrete a substance of interest comprises culturing the cells in the adequate conditions to allow binding of the substances of interest secreted to the medium to the functionalizing molecule.

The expression “Culturing the cells in the adequate conditions to allow binding of the substances of interest secreted to the medium to the functionalizing molecule” refers to the process of maintaining the cell population viability by supplying the cells with adequate culture media as well as support media additives and placing the cell population under the correct temperature, humidity, light and agitation conditions for the cells to remain viable. Examples of culture media and support media additives have been mentioned previously and are equally valid for the current aspect. By maintaining the cell population viability, the cells will continue to produce and secrete the substance of interest which will then bind to the functionalizing molecule.

In a preferred embodiment of the secretion method of the invention the functionalizing molecule is selected form a group consisting of: antibody, any of the functionalizing molecules of Table 1 and any combination thereof.

TABLE 1
List of functionalizing molecules for the secretion method of the invention
together with the substance which can generates a detectable signal.

| | Substance | |
|---|---|---|
| | of interest | |
| Functionalizing molecule | detected | References |
| Glucose-Responsive | Glucose | Shibata et al., 2010, PNAS 107 (42) |
| Monomer | | 17894-17898 |
| Glucose oxidase | Glucose | Kim et al., 2016, Polym. Chem., 7, |
| | | 1907-1912 |
| Lactate oxidase | Lactate | Zhang et al., 2017, ACS Appl. Mater. |
| | | Interfaces 9, 44, 38153-38158 |
| Extracellular Oxygen | O₂ | Zhu et al., 2019, Theranostics May |
| Consumption Reagent | | 18; 9(11): 3293-3307 |
| Pt-porphyrin | O₂ | Saito et al., 2007, Journal of Porphyrins |
| | | and Phthalocyanines, 11, 03, pp. 160-164 |
| 4,4-difluoro-4-bora-3a,4a- | Ca²⁺ | Sui et al., 2016, Chemistry 18, 22(30): |
| diaza-s-indacene(BODIPY)- | | 10351-4 |
| based fluorophore and a Ca2+ | | |
| recognition group | | |
| Solvatochromic Probe | CO₂ | Ali et al., 2011, Anal. Chem., 83, 8, |
| | | 2846-2851 |
| Dicationic | CO₂ | Mulugeta et al., 2017, Chem 3, 1008- |
| mesoBis(benzimidazolium) | | 1020 |
| Calix[4]pyrrole | | |
| H₂O₂-selective probes | H₂O₂ | Grisham, 2013, Comp Biochem Physiol |
| | | A Mol Integr Physiol August; 165(4): 429-38. |
| Sensing Indicators | Na⁺ and K⁺ | https://www.thermofisher.com/es/es/home/ |
| | | references/molecular-probes-the-handbook/ |
| | | indicators-for-na-k-cl-and-miscellaneous- |
| | | ions/fluorescent-na-and-k-indicators.html |
| | | (accessed on the 30 Jul. 2021) |
| Aggregation-Induced | Heavy | Neupane et al., 2016, Anal. Chem., 88, 6, |
| Emission | metals | 3333-3340 |
| FRET | pH | Liu et al., 2020, Front. Bioeng. |
| | | Biotechnol., Nov 9;8:595497 |
| Fluorescence Probes | pH | Chen, 2021, Anal Biochem, Jan |
| | | 1;612:113900 |

As used herein, the term “antibody” refers to monoclonal antibodies (mAbs), multispecific antibodies, human antibodies, humanized antibodies, synthetic antibodies, chimeric antibodies, polyclonal antibodies, camel antibodies, single chain FVS antibodies, single-chain antibodies, immunologically active antibody fragments (e.g., antibody fragments capable of binding epitopes, e.g., Fab fragments, Fab′ fragments, F(AB′)2 fragments, Fv fragments, fragments containing a VL or VH domain or complementarity determining regions (CDRs) immunospecifically bind antigen, etc.), bi- or poly-functional antibodies, disulfide-linked bispecific FVS (sdFv), intrabody and diabodies, and any of the above epitope binding fragments. In particular, the term “antibody” is intended to include immunologically active fragments of immunoglobulin molecules and immunoglobulin molecules, i.e., molecules containing an antigen binding site. Immunoglobulin molecules can be of any type (e.g., IgG, IgE, IgM, IgD, IgA, and IgY), class (e.g., IgGbIgG2, IgG3, IgG4, IgAi, and IgA2) or subclass.

In a third step, the method for determining if a cell population secretes to the medium a substance of interest which comprises detecting the binding of the substance of interest to the molecule of interest by detecting the signal resulting from said interaction.

The expression “Detecting the binding of the substance of interest to the molecule of interest by detecting the signal resulting from said interaction” as used herein refers to the process of identifying the binding of the substance of interest to the functionalizing molecule. Said detection may be a direct detection of the substance of interest through the use of molecules which bind to it, such as aptamers, such as structure switching signaling aptamers (SSSA) or antibodies, such as primary antibodies anti-substance of interest labelled or a conjugation of primary antibodies anti-substance of interest with secondary antibody anti primary antibody, wherein the secondary antibody is labeled with a detectable marker. Detection may be performed trough optical microscopy or fluorescence microscopy.

### Method for Determining the Effect of an Effector Molecule on a Cell Population and for Determining if the Cell Population Secretes to the Medium a Substance of Interest

The authors of the present invention have found that, by combining in the same support microbeads containing functionalizing molecules with a effector function on the cell population within the support and microbeads containing functionalizing molecules capable of interacting with a substance of interest, the support of the present invention allows the simultaneous detection of an effect of a functionalizing molecule on a cell population and the production of a cell of interest by said cell population.

Another aspect of the present invention relates to a method for determining the effect of an effector molecule on a cell population and for determining if the cell population secretes to the medium a substance of interest, from here on the dual method of the invention, which comprises the steps of:
(i) Providing a support according to the invention wherein said support contains the cell population, wherein the cells of the population are attached to the support and wherein the first and second type of microbeads contain, respectively, a first type of functionalizing molecule which is the effector molecule and a second type of functionalizing molecule which is capable of interacting with the substance of interest, said interaction resulting in a detectable signal;
(ii) Culturing the cells under conditions adequate to allow the effector molecule to exert its effect on the cells of the cell population and the binding of the substances of interest secreted to the medium to the detector molecule; and
(iii) Detecting the response of the cells within the cell population to said effector molecule and detecting the binding of the substance of interest to the detector molecule by detecting the signal resulting from said interaction.

All the previous defined terms and expressions are equally valid for the present aspect and its embodiments.

In a preferred embodiment of the dual method of the invention, the support contains at least two types of the first type of microbeads and at least two types of the second type of microbeads, wherein each type of microbead of the first type is modified with at least one functionalizing molecule, and each type of microbead of the second type is modified with at least one functionalizing molecule, wherein the functionalizing molecules are identical or different between the different types of microbeads.

In another embodiment of the dual method of the invention the effector molecule is selected from a group consisting of growth factors, differentiation factors, cell adhesion molecules or proteins, pharmaceutical small molecules and any combination thereof.

In yet another embodiment of the dual method of the invention, the growth factor is selected from the group consisting of: vascular endothelial growth factor (VEGF), collagen, bone morphogenic factor-β, epidermal cell growth factor (EGF), platelet derived growth factor (PDGF), nerve growth factor (NGF), fibroblast growth factor (FGF), insulin-like growth factor (IGF), transforming growth factor (TGF) and any combination thereof.

In an additional embodiment of the dual method of the invention the substance of interest of step (i) is selected from a group consisting of: exosomes, extracellular vesicles, lipoproteins, ferritin, viruses, human growth hormone, N-methionyl human growth hormone, bovine growth hormone, parathyroid hormone, thyroxine, insulin, proinsulin, relaxin, prorelaxin, glycoprotein, follicle stimulating hormone (FSH), thyroid stimulating hormone (TSH), luteinizing hormone (LH), hepatocyte growth factor, fibroblast growth factor, prolactin, placental lactogen, factor-α, tumor growth-β, Mullerian inhibiting substance, gonadotropin-associated peptide mouse, inhibin, activing, vascular endothelial growth factor, integrin, thrombopoietin (TPO), EGF-β growth, platelet growth factor, transforming growth factor (TGF)-α, TGF-β, insulin-like factor-I growth, insulin-like factor-II growth, Erythropoietin (EPO), osteoinductive factor, interferon-α, interferon-β, interferon-γ, macrophage-CSF (M-CSF), CSF granulocyte-macrophage (GM-CSF), granulocyte-CSF (G-CSF) interleukin (IL)-1, IL-la, IL-2, IL-3, IL-4, IL-5, IL-6, IL-7, IL-8, IL-9, IL-11, IL-12; tumor necrosis factor (TNF)-α, TNF-β, leukemia inhibitory factor (LIF), kit ligand (KL), glucose, lactate, oxygen (O₂), calcium (Ca²⁺), carbon dioxide (CO²⁺), Hydrogen peroxide (H₂O₂), sodium (Na⁺), potassium (K⁺), cadmium (Cd²⁺), cobalt (Co²⁺), palladium (Pb²⁺), copper (Cu²⁺), silver (Ag), magnesium (Mg²⁺), manganese (Mn²⁺), nickel (Ni²⁺), zinc (Zn²⁺), chromium (Cr³⁺), iron (Fe³⁺), aluminum (Al³⁺), mercury (Hg²⁺) and any combination thereof.

On more preferred embodiment of the dual method of the invention the effect of the effector molecule on the cell population is the secretion of the substance of interest.

The invention is also defined by way of the following aspects:
1. A support suitable for culturing cells characterized in that it contains microbeads attached to its surface wherein the microbeads are modified with at least one functionalizing molecule, the microbeads are uniformly distributed in a predetermined pattern within the support and the microbeads define regions in the support which show reduced adhesive capacity to cells with respect to the regions onto which the microbeads are not attached.
2. The support according to aspect 1 wherein the predetermined pattern of microbeads is created by allowing the microbeads to adhere to the support when the support is in contact with a stamp, said stamp having protrusions which define a pattern which is the negative of the predetermined pattern, thereby preventing the microbeads from attaching to those areas of the support which are in contact with the stamp protrusions.
3. The support according to aspects 1 or 2 wherein the regions in the support onto which the microbeads are not attached are coated with a compound or composition that promotes cell adhesion.
4. The support according to aspect 3 wherein the coating of the support with a compound or composition that promotes cell adhesion is carried out by coating the protrusions of the stamp with said compound or composition that promotes cell adhesion and contacting the support with the stamp thereby allowing the transfer of the compound or composition from the stamp onto the support with a pattern that matches the pattern of protrusions of the stamp.
5. The support according to any of aspects 1 to 4 wherein the regions in the support onto which the microbeads are attached are further coated with a compound or composition which substantially reduces cell adhesion.
6. The support according to any of aspects 1 to 5 where in the support is made from a material selected from a group consisting of: glass, borosilicate glass, quartz glass, polystyrene, polymethylmethacrylate (PMMA), polycarbonate, polyethylene and cyclic olefin copolymers, preferably glass.
7. The support according to any of aspects 2 to 6 wherein the predetermined pattern of microbeads is created using vacuum-driven soft lithography.
8. The support according to any of aspects 1 to 7 wherein the compound that promotes cell adhesion is an extracellular matrix molecule, preferably fibronectin.
9. The support according to any of aspects 1 to 8 wherein the support further contains viable cells which are attached to the support by adhesion to the compound which promotes cell adhesion.
10. The support according to any of aspects 1 to 9 wherein the predetermined pattern has a geometrical form, preferably a circular form with a diameter of between 10 μm to 200 μm.
11. The support according to any one of aspects 1 to 10 wherein the microbeads are selected from a group consisting of: glass microbeads, polystyrene microbeads, poly (methyl methacrylate) microbeads, silica microbeads, zirconia microbeads, titanium microbeads, gold microbeads, polyethylenepolylactic acid (PLA) microbeads, poly-L-lactic acid (PLLA) microbeads, poly glycolic acid (PGA) microbeads, poly lactic-co-glycolic acid (PLGA) microbeads and poly-caprolactone (PCL) microbeads, preferably polystyrene microbeads.
12. The support according to any one of aspects 1 to 11 wherein the diameter of the microbeads is between 50 nm and 25000 nm.
13. The support according to any one of aspects 1 to 12 wherein the functionalizing molecule is attached to the microbeads through a coupling molecular pair, wherein one member of the coupling molecular pair is bound to the microbeads and the other member of the coupling molecular pair is bound to the molecule of interest.
14. The support according to aspect 13 wherein the coupling molecule pair is the streptavidin-biotin molecular pair.
15. A method for producing the support according to any of aspects 1 to 14 comprising the steps of:
   (i) Providing a support;
   (ii) Contacting the support with a stamp, said stamp having protrusions which define a pattern which is the negative of the predetermined pattern, thereby preventing the microbeads from attaching to those areas of the support which are in contact with the stamp protrusions, wherein the contact of the support with the stamp forms bottomless channels between the support and the stamp;
   (iii) Contacting the support with the microbeads along the bottomless channels and allowing the microbeads to adhere to the support as to obtain a uniform deposition of microbeads in the predetermined pattern within the support thereby defining regions in the support which show reduced adhesive capacity to cells with respect to the regions onto which the microbeads are not attached.
16. The method according to aspect 15 wherein the regions in the support onto which the microbeads are not attached are coated with a compound or composition that promotes cell adhesion.
17. The method according to aspect 16 wherein the coating of the support with a compound or composition that promotes cell adhesion is carried out by coating the protrusions of the stamp of step (ii) with said compound or composition that promotes cell adhesion thereby allowing the transfer of the compound or composition from the stamp onto the support with a pattern that matches the pattern of protrusions of the stamp.
18. The method according to aspect 15 wherein the regions in the support onto which the microbeads are attached in step (iii) are further coated with a compound or composition which substantially reduces cell adhesion.
19. The method according to any of aspects 15 to 18 wherein the support of step (i) is made from a material selected from a group consisting of: glass, borosilicate glass, quartz glass, polystyrene, polymethylmethacrylate (PMMA), polycarbonate, polyethylene and cyclic olefin copolymers, preferably glass.
20. The method according to aspects 15 or 19 wherein step (ii) is carried out vacuum-driven soft lithography.
21. The method according to any of aspects 15 to 20 wherein the microbeads of step (iii) are selected from a group consisting of: glass microbeads, polystyrene microbeads, polyacrylamide microbeads, poly (methyl methacrylate) microbeads, silica microbeads, zirconia microbeads, titanium microbeads, gold microbeads, polyethylenepolylactic acid (PLA) microbeads, poly-L-lactic acid (PLLA) microbeads, poly glycolic acid (PGA) microbeads, poly lactic-co-glycolic acid (PLGA) microbeads and poly-caprolactone (PCL) microbeads, preferably polystyrene microbeads.
22. The method according to any of aspects 15 to 21 wherein the microbeads of step (iii) have a diameter between 50 nm and 25000 nm.
23. The method according to any of aspects 15 to 22 wherein the stamp is a PDMS stamp.
24. The method according to any of aspects 15 to 23 wherein the chamber formed between the stamp and the support is provided with an operable inlet and an operable outlet and wherein step (iii) is carried out by injecting a suspension of microbeads through the inlet while the outlet is closed, maintaining the suspension within the channels for a sufficient time so as to obtain a uniform deposition of microbeads on the support and extracting the suspension through the outlet.
25. The method according to aspect 24 wherein step (iii) is carried out while pressure is being applied to bring into contact the slab and the support.
26. The method according to aspect 25 wherein the pressure is achieved by creating vacuum in the chamber formed between the support and the slab.
27. The method according to any of aspects 25 to 26 wherein the suspension of microbeads further comprises a compound or composition which substantially reduces cell adhesion.
28. The method according to any of aspects 15 to 27 further comprising contacting the support with a cell population under conditions adequate for the binding of the cells to the support through interactions between the cells and the compound in the support that promotes cell adhesion.
29. The method according to aspect 28 wherein the cell population is placed inside a well-like structure wherein said well-like structure is formed by a base and vertical walls wherein the base is formed by the support layer comprising the predetermined patterns base.
30. The method according to aspect 29 wherein the well-like structure vertical walls are made from a material selected from a group consisting of: polydimethylsiloxane (PDMS), glass, poly (methyl methacrylate) (PMMA), pressure sensitive adhesive sheets (PSA), cyclic olefin copolymer (COC), cyclic olefin polymers (COP), polyethylene terephthalate (PET), BioMed Clear Resin (Formlabs), polyether ether ketone (PEEK), polystyrene (PS), polypropylene (PP), polyethylene (PE), polycarbonate or any combination thereof.
31. A method for determining the effect of a molecule on a cell population which comprises the steps of:
   (i) Providing a support according to any of the aspects 1 to 14 wherein said support contains the cell population wherein the cells of the population are attached to the support and wherein the molecule, the effect of which on the cell population is to be determined is the functionalizing molecule;
   (ii) Maintaining the support under adequate conditions to allow the functionalizing molecule to exert its effect on the cells of the cell population; and
   (iii) Detecting the response of the cells within the cell population to said functionalizing molecule,
      wherein the detection of a response of the cells to the functionalizing molecule is indicative that the functionalizing molecule exerts an effect on the cells of the cell population.
32. The method according to aspect 31 wherein the functionalizing molecule is selected from a group consisting of growth factors, differentiation factors, cell adhesion molecules or proteins, pharmaceutical small molecules and any combination thereof.
33. The method according to aspect 32 wherein the growth factor is selected from the group consisting of: vascular endothelial growth factor (VEGF), collagen, bone morphogenic factor-β, epidermal cell growth factor (EGF), platelet derived growth factor (PDGF), nerve growth factor (NGF), fibroblast growth factor (FGF), insulin-like growth factor (IGF), transforming growth factor (TGF) and any combination thereof.
34. A method for determining if a cell population secretes to the medium a substance of interest which comprises the steps of:
   (i) Providing a support according to any of the aspects 1 to 14 wherein said support contains a cell population wherein the cells of the population are attached to the support and wherein the functionalizing molecule comprises a molecule which is capable of interacting with the substance of interest, said interaction resulting in a detectable signal;
   (ii) Culturing the cells in the adequate conditions to allow binding of the substances of interest secreted to the medium to the functionalizing molecule; and
   (iii) Detecting the binding of the substance of interest to the molecule of interest by detecting the signal resulting from said interaction.
35. The method according to aspect 34 wherein the substance of interest of step (i) is selected from a group consisting of: exosomes, extracellular vesicles, lipoproteins, ferritin, viruses, human growth hormone, N-methionyl human growth hormone, bovine growth hormone, parathyroid hormone, thyroxine, insulin, proinsulin, relaxin, prorelaxin, glycoprotein, follicle stimulating hormone (FSH), thyroid stimulating hormone (TSH), luteinizing hormone (LH), hepatocyte growth factor, fibroblast growth factor, prolactin, placental lactogen, factor-α, tumor growth-β, Mullerian inhibiting substance, gonadotropin-associated peptide mouse, inhibin, activing, vascular endothelial growth factor, integrin, thrombopoietin (TPO), EGF-β growth, platelet growth factor, transforming growth factor (TGF)-α, TGF-β, insulin-like factor-I growth, insulin-like factor-II growth, Erythropoietin (EPO), osteoinductive factor, interferon-α, interferon-β, interferon-γ, macrophage-CSF (M-CSF), CSF granulocyte-macrophage (GM-CSF), granulocyte-CSF (G-CSF) interleukin (IL)-1, IL-la, IL-2, IL-3, IL-4, IL-5, IL-6, IL-7, IL-8, IL-9, IL-11, IL-12; tumor necrosis factor (TNF)-α, TNF-β, leukemia inhibitory factor (LIF), kit ligand (KL), glucose, lactate, oxygen (O₂), calcium (Ca²⁺), carbon dioxide (CO²⁺), Hydrogen peroxide (H₂O₂), sodium (Na⁺), potassium (K⁺), cadmium (Cd²⁺), cobalt (Co²⁺), palladium (Pb²⁺), copper (Cu²⁺), silver (Ag⁺), magnesium (Mg²⁺), manganese (Mn²⁺), nickel (Ni²⁺), zinc (Zn²⁺), chromium (Cr³⁺), iron (Fe³⁺), aluminum (Al³⁺), mercury (Hg²⁺) and any combination thereof.

The invention will be described by way of the following examples which are to be considered as merely illustrative and not limitative of the scope of the invention.

### EXAMPLES

### Example 1: Method for Determining the Effect of the Functionalizing Molecule on a Cell Population

### Example 1.1: Materials and Methods

### Materials

Bovine plasma fibronectin was purchased from Fisher Scientific, Spain. Bovine serum albumin (BSA) was purchased from Sigma Aldrich, Spain. Polydimethylsiloxane silicone elastomer and curing agent 184 were purchased from Ellsworth adhesives, Spain. Streptavidin coated polystyrene microbeads (400 nm Ø) were purchased from Immunostep, Spain. Biotinylated FGF-2 (listed as FGF-2 in the manuscript) was purchase from Deltaclon, Spain. FGFR-1 rabbit polyclonal antibody and Goat anti-Rabbit IgG Alexa Fluor 647 antibody were purchased from Fisher Scientific, Spain. Polymethyl methacrylate (PMMA) Plexiglas 4 mm, was purchased from Evonik Industries AG, Germany. Pressure Sensitive Adhesive (PSA) ARcare 8939 was purchased from Adhesive Research, Ireland. Human adult Mesenchymal Stromal Cells were obtained from human follicles (hHF MCSs, p4). Complete Medium (CM) consisted of Dulbecco's Modified Eagle's Medium (DMEM) (Fisher Scientific Spain) supplemented with 30% Fetal Bovine Serum (FBS) (Fisher Scientific Spain) and 10% Penicillin/Streptomycin (P/S) (Fisher Scientific Spain). Serum-free DMEM medium (SFM) FBS consisted in Dulbecco's Modified Eagle's Medium (DMEM) (Fisher Scientific Spain) with 10% Penicillin/Streptomycin (P/S) (Fisher Scientific Spain). Paraformaldehyde 4% was purchased from Panreac Quimica, Spain.

### Fabrication of PDMS Slabs

In order to characterize the deposition of the microbeads and generate combined patterns of cells and microbeads, several types of empty channel-like structures and channel-like structures containing micropillars were designed. Briefly, all structures were designed using CleWin software and adapted into a photomask. The obtained photomask were transferred to silicon wafers through photolithography using SU8 resin for formation of the features, obtaining structures containing hills and valleys corresponding to the channel-like structures and the pillars structures, respectively.

The silicon wafers served as masters for the generation of the polydimethylsiloxane (PDMS) slabs. 40 mL of PDMS (silicone elastomer and curing agent ratio 10:1, respectively), were poured on top of the masters and polymerized at 70° for 2 h. The resulting PDMS slabs contained either bottom-less channels with different lengths, widths and heights, or bottom-less channels containing small pillars of 50 and 100 μm diameter separated of each other by 100 μm.

### Patterning of Polystyrene Microbeads

Deposition and patterning of streptavidin coated polystyrene microbeads was carried out through the method of vacuum-driven soft lithography. In all cases, PDMS slabs channels were punched twice in order to generate a 2 mm diameter inlet and a 1 mm outlet. Glass covers (24×60 mm) were carefully rinsed with ethanol and distilled water and were dried with compressed air. PDMS slabs were put in close contact with the glass slides, serving the glass as the bottom of the channels, enclosing them. The resulted pasted PDMS slabs on glass were put under vacuum inside of a desiccator for 30 minutes. Afterwards, the outlets were plugged with tape and 2 μL of microbeads suspension was loaded on the inlets. Outlet was required to avoid the phase separation during flow, which interferes with the distribution of the suspension inside the channel. Closing of the outlet was required to maintain the passive pumping inside the channel. Suspension was let flow until it started filling the outlet. Tape was removed after 5 minutes and PDMS slabs-glass covers were left overnight for solvent evaporation at 4° C. Finally, PDMS slabs were removed afterwards.

Co-Patterning of Microbeads and hHF-MSCs

In order to generate combined patterns of microbeads and hHF-MSCs, Printing and Vacuum Lithography (PnV Litho) was carried out. PDMS slabs channel-like structures (1000×5000×13 μm, width·length·height) with pillars inside (50 or 100 μm diameter, Slab50 and Slab 100) were punched as previously explained. Pillars inside of the channels were wetted with a solution of fibronectin 50 μg mL⁻¹ for 30 minutes. Afterwards, PDMS slabs were rinsed with distilled water, dried with compressed air and attached to PMMA wells with glass bottom. Subsequently, the vacuum lithography protocol was followed. The concentration of microbeads used in all cases was 8×1010 microbeads mL⁻¹. Upon solvent evaporation and PDMS removal, patterns of fibronectin dots (either 50 or 100 μm) surrounded by microbeads were obtained. Finally, wells were loaded with 1 mL of BSA solution 5% as a blocking agent.

To quantify the time required to fully occupy all the dots in a pattern, hHF-MSCs were detached from the flasks and were resuspended in serum free medium (SFM) at a concentration of 100000 cells·mL⁻¹. 750 μL of the cell suspension were loaded into the PMMA wells previously modified to contain patterns of fibronectin dots (either 50 or 100 μm, D₅₀ and D₁₀₀) surrounded by microbeads, and were left inside the incubator on constant oscillation in a rocker (Vari-Mix steep angle rocker, Thermo Fisher) for 30, 60 or 120 minutes (n=3 per experimental condition). Afterwards, medium was retrieved, wells were rinsed three times with PBS to wash out any non-attached cell and patterned cells were fixed with paraformaldehyde 4%. Afterwards, cells were photographed and counted.

To evaluate the direct FGF-2-driven stimulation of patterned hHF-MSCs, microbeads were functionalized with FGF-2. Briefly, stock microbeads suspension were centrifuged at 6000 rpm for 9 minutes. Afterwards, microbeads were resuspended with either 10 μL of FGF-2 10 μg mL⁻¹ or 10 μL of distilled water, and were left incubating at room temperature for 30 minutes. Afterwards, suspensions were centrifuged in the same conditions. Finally, both suspensions were resuspended in distilled water for a final concentration of 8×1010 microbeads mL⁻¹. In total, three different batch of FGF functionalized microbeads were made: one containing exclusively FGF-2 functionalized microbeads (100% FGF-2), one with non-treated microbeads (0% FGF-2) and one with a mixture 1:1 of both batches (50% FGF-2). Three samples were done per experimental condition. PnV Litho was carried out with all three batches using PDMS Slab₅₀ and Slab100 on PMMA wells with glass bottom. Upon solvent evaporation, all wells were blocked with 1 mL of 5% BSA solution. Subsequently, wells were loaded with 750 μL of hHF-MSCs suspension (p4) 10⁵ cells mL⁻¹ in SFM and left inside the incubator for 2 hours on constant oscillation in a rocker. Afterwards, medium was retrieved from all wells. Wells were rinsed three times with PBS and replenish with 750 μL of fresh SFM. All samples were left inside the incubator for 24 hours. At the end of the experiment, cells were photographed and counted.

### Immunocytochemistry of FGFR-1

In order to verify the enhanced expression of FGFR-1 with direct FGF stimulation, microbeads and hHF-MSCs were patterned with the PnV Litho methodology. In this case, PDMS Slab100 were used. hHF-MSCs small cell-colonies were co-patterned either with 50% FGF-2 or 0% FGF-2 microbeads and left incubating for 4 hours. Afterwards, cell culture medium was retrieved, wells were rinsed three times with PBS and cells were fixed with paraformaldehyde 4% for 10 minutes. Three samples were done per experimental condition.

Afterwards, samples were incubated with a BSA solution 1% for 1 h and were subsequently incubated with a solution of primary antibody containing FGFR-1 rabbit antibody 5 μg mL⁻¹, BSA 0.2% (w/v) and Goat Serum 1% (v/v) for 2 hours. Samples were rinsed with PBS three times and were incubated with a secondary antibody solution containing Goat anti-rabbit IgG Alexa Fluor 647 5 μg mL⁻¹, BSA 0.2% (w/v) and Goat Serum 1% (v/v) for 45 minutes. Finally, samples were rinsed and analyzed.

### Image and Data Analysis

Brightfield and fluorescence microscope images were taken with a modified Nikon Eclipse TE2000-S inverted microscope (USA), with and adapted Andor Zyla sCMOS black and white camera (Oxford Instruments, UK). Lumencor laser 640 nm was used as light source for excitation and Quad EM filter: 446/523/600/677 with 4 TM bands: 446/34+523/42+600/36+677/28. Microscopy images were processed by FiJi/ImageJ software.

### Example 1.2: Results and Discussion

Co-Patterning of Microbeads and hHF-MSCs

First, combined patterns of microbeads and adhesion proteins were generated by printing and Vacuum lithography (PnV Litho). The deposition of the streptavidin coated microbeads on glass surfaces was tested to determine the minimum microbeads concentration and channels designs needed to produce a homogeneous and reproducible distribution of microbeads on the glass substrates. In summary, microbeads suspension concentration greatly affected the density and distribution of the microbeads on the glass surface, where microbeads suspensions with higher concentrations resulted in higher density of microbeads in the surface. Increasing the channel's length (from 0.5 cm to 1 cm and 2 cm) and height (from 13 μm to 25 μm) resulted in a decrement of the microbeads density on the surface. Finally, changing the channel's width did not produce any significant differences in the density and distribution of the microbeads on the glass surface.

Afterwards, we performed the combined patterning of microbeads and fibronectin dots by PnV Litho, and the subsequent patterning of cells to produce a small cell-colonies array where each colony could be surrounded by microbeads. PDMS slabs with microchannels (1000×5000×13 μm, width×length×height) were fabricated containing two types of micropillars arrays inside each channel; one with 50 μm diameter pillars separated by 100 μm (222 pillars per array), and another with 100 μm diameter pillars separated by 100 μm from each other (125 pillars per array). For the sake of clarity, the two different PDMS microchannel designs and the patterns obtained with both types of channels will be called Slab₅₀ Slab100 and D₅₀ and D₁₀₀ substrates, respectively.

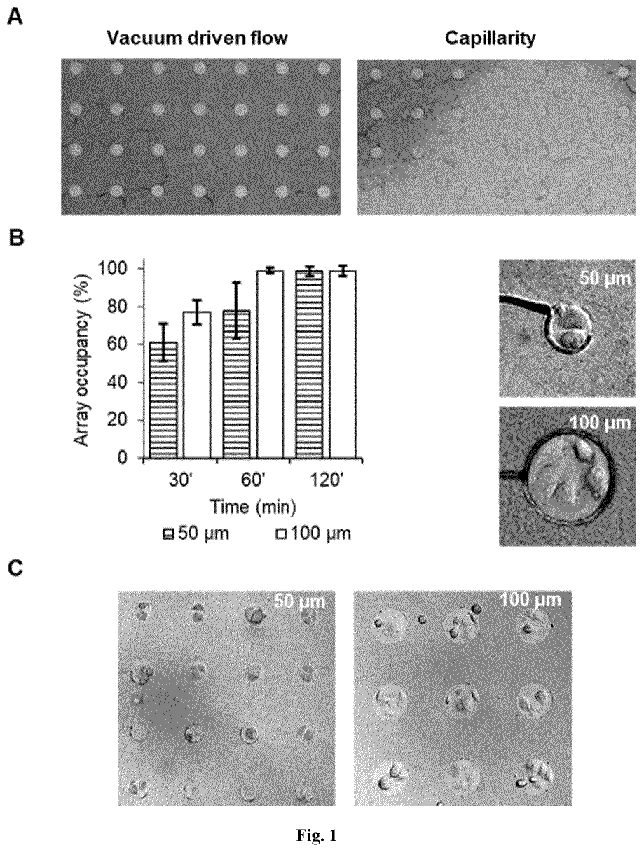

The PDMS Slab₅₀ and Slab₁₀₀ were inked with fibronectin, assembled and put in vacuum. Afterwards, a suspension of microbeads (8×10¹⁰ microbeads mL⁻¹) was loaded in the microchannels and let dry overnight. The result was a good pattern of microbeads surrounding the fibronectin protein dots on the glass substrates, as revealed by brightfield microscopy (FIG. 1A). The PnV Litho was an optimal technique for the preparation of multicomponent patterns on glass. It did not require external pumps to generate a flow of microbeads suspension in the PDMS stamp and when compared with capillarity filling, the sucking generated from the PDMS matrix improved the homogenous distribution of the liquid inside. Higher density of microbeads could be observed in the contact line between the protein dot and the particles pattern surrounding each spot due to the sucking effect produced in each pillar in the PDMS slab. In some cases, higher accumulation of microbeads could be observed between the dots.

D₅₀ and D₁₀₀ patterns were then incubated with an hHF-MSCs suspension to form pattern of small cell-colonies surrounded by microbeads. As seen in FIG. 1, Brightfield microscopy showed a good coverage of the glass substrate with cells and cells adhered specifically on the protein dots previously printed. The size of the protein dots of the arrays D₅₀ and D₁₀₀ determined the number of cells in each colony and the time to fill the array. For the D₅₀ substrates, the number of cells attached to each dot varied between 1 to 3 cells, with a mean number of 1.95±0.10 cells per dot. The total amount of cells for the D₅₀ was approximately 430+26 cells per sample. For the D₁₀₀ substrates, higher variability of cells per dot could be found, ranging from 2 to 9 cells in a single dot. However, the majority of dots contained between 4 and 5 cells, with a mean number of 4.20±0.5 cells per dot. The total amount of cells for the D₁₀₀ was approximately 530±65 cells per sample.

To study the time required to obtain a full array occupancy (where all fibronectin dots of an array are occupied by cells), hHF-MSCs were incubated with the both D₅₀ and D₁₀₀ substrates for different time lapses (30, 60 and 120 minutes). In all cases, there was higher array occupancy with higher incubation times. As seen in FIGS. 1B and C, the total array occupancy for the D₅₀ substrates was obtained after 120 minutes incubation. For the D₁₀₀ substrates, the total array occupancy was obtained at 60 minutes.

These results demonstrated the capability of the PnV Litho for the creation of an array of cells surrounded by patterned microbeads. The array was comprised of high number of independent events, each one of them serving as their own independent experimental replica, with controlled chemical interactions with their surface and controlled cell-cell contact and cell adhesion.

Direct FGF-2-Driven Stimulation of hHF-MSCs Arrays

Co-patterned FGF-2 functionalized microbeads and hHF-MSCs D₅₀ and D₁₀₀ substrates were used to evaluate the FGF-2-driven stimulation on the cell's proliferation. The microbead suspensions loaded were either composed of non-treated microbeads (0% FGF-2, bare microbeads), fully FGF-2 functionalized microbeads (100% FGF-2), or a mixture 1:1 of the other two (50% FGF-2). This way, two different cell-cell contact and cell confluence scenarios were exposed to three different dosages of FGF-2. Cells were loaded to each substrate and the resulting patterned cells were incubated for 24 hours in intimate contact with the microbeads. Afterwards, cells were photographed through brightfield microscopy.

The presence of FGF-2 resulted in higher number of cells per array when compared with 0% FGF-2.

The total number of cells remaining in D₅₀ substrates decreased during the 24 hours. This is a result of the apoptotic effect produced by the patterning conditions. However, significant differences in the cell number could observed between both patterns with 0% and 50% FGF-2 microbeads.

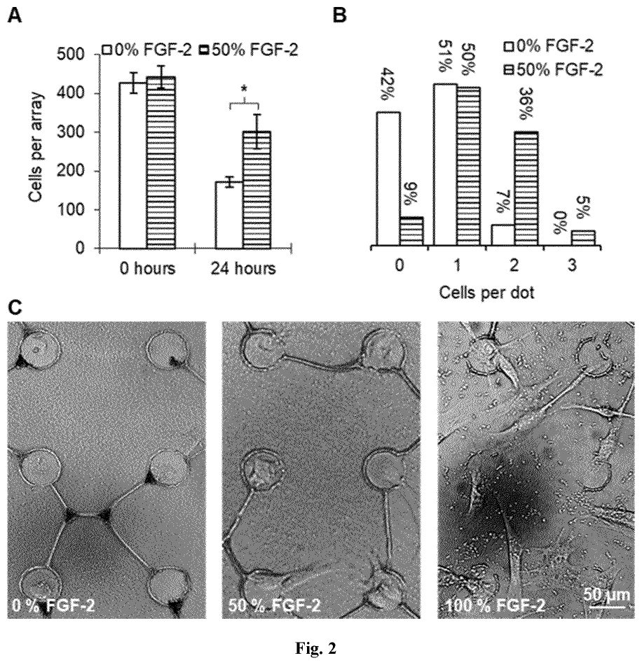

The final number of cells per array was 76% higher in the cells co-patterned with 50% FGF-2 microbeads than those patterned and incubated with 0% FGF-2 microbeads, see FIG. 2A. Differences were observed both in the number of cells per dot and the total number of dots occupied, FIG. 2B. In the case of the bare 0% FGF-2 microbeads patterns, 42% of the dots were empty and 51% of the dots were occupied by only 1 cell after 24 h. For the cells co-patterned with 50% FGF-2 microbeads, 50% and 36% of the dots were occupied by one or two cells respectively.

Regarding cell's morphology, cells treated with FGF-2 presented a more elongated type of morphology, FIG. 2C. While most cells were round in the 0% FGF-2 patterns at 24 hours, cells in the 50% FGF-2 patterns actually elongated and partially grew outside of their dot. All this indicated an effective interaction with the FGF-2 functionalized microbeads. In the case of the cells patterned with 100% FGF-2 functionalized microbeads, all cells grew and migrated outside of the boundaries of their respective dot, destroying the array, indicating a higher effect due to a higher dose of FGF-2 in contact with the cells. This effect can be attributed to the higher dose of FGF-2 loaded in the 100% FGF-2 microbeads patterns. The movement generated by the cells also destroyed the microbeads pattern, dispersing it along the substrate, even adhering some microbeads to the cells surface.

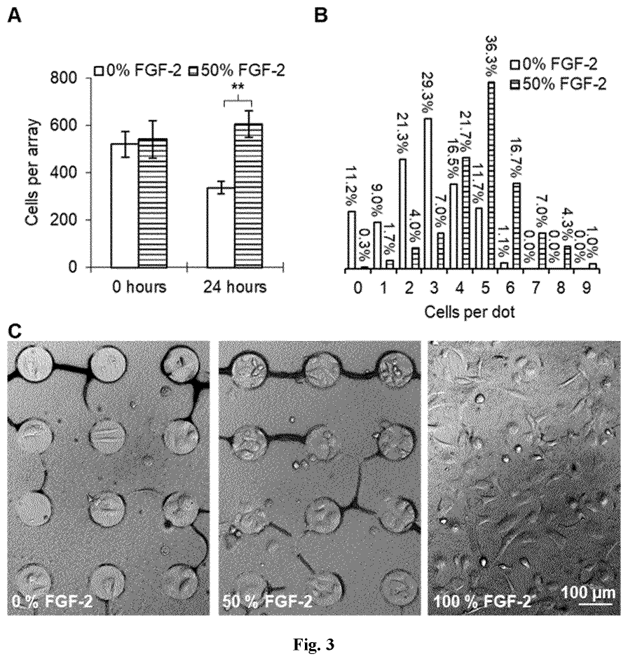

The total number of cells in D₁₀₀ substrates augmented in 24 hours when patterned with 50% FGF-2. The number of cells per array in the 50% FGF-2 microbead arrays was 80% higher than the ones co-patterns with bare 0% FGF-2 microbeads, see FIG. 3A. Regarding the number of cells per dot, as seen in FIG. 3B, the arrays with 0% FGF-2 microbeads showcased a decrement of 35% cells when compared to the number of cells at time 0, where 11% of the dots were empty and 21% and 29% of the dots had two or three cells, respectively. In the arrays with 50% FGF-2 microbeads, the number of cells increased by 11% in 24 h, where 99% of the dots were occupied by one or more cells and 21% and 36% of the dots had four or five cells, respectively.

In all scenarios, cells remained as elongated as the spot allowed them. For the cells co-patterned with 50% FGF-2 microbeads, some cells also appeared to grow outside of the boundaries of the dots, as seen in the D50 substrates. In the 100% FGF-2 microbeads patterns, the same effect observed for the D50 substrates occurred, in which cells grew completely out of the array, also breaking the microbeads pattern, see FIG. 3C.

In the case of D₁₀₀ substrates, in contrast with D₅₀ substrates, the total cell number augmented in 24 hours when in presence of FGF-2, indicating that cell-cell interactions and cell density can affect the stimulation produced by FGF-2, where the higher cell density may serve as protection from the effects apoptotic effects produced by the patterning conditions.

Taking all into account, D₅₀ and D₁₀₀ not only allowed a more controlled dosage of FGF-2 to each small cell-colony but also improved the effect on cell's survival and proliferation (76% and 80% more cells after 24 h for the 50% FGF-2 microbead arrays when compared with the 0% FGF-2 arrays) over more conventional methodologies. When stimulating cultured hHF-MSCs on well plates with dissolved FGF-2 (10 ng mL-1 40) on the same conditions, only 58% more cells could be observed in the samples incubated with FGF-2 after 48 h, indicating less proliferation effect in twice the time. When functionalizing directly the bottom surface of the well plates with FGF-2, not only less proliferation could be observed (60% more cells in the functionalized well plates when compared with the non-treated samples after 24 h), but a heterogeneous distribution of the adhered cells could be observed as a result of the uncontrolled deposition of the growth factor on the surface. Finally, when adhering cells directly on top of 100% FGF-2 microbead patterns, 63% more cells could be observed in the samples with functionalized microbeads when compared with the samples containing 0% FGF-2 after 24 h, indicating that the control of cell adhesion and the control of cell-cell contact also influence an enhances the effect FGF-2.

These results falls in accordance with the current knowledge that the control of spatial distribution of the growth factors on solid-phase presentation systems promotes the efficiency of the stimulation.

In all, our tunable platform allowed the generation of dozens of individual cell colony replicas with control over cell-cell and cell material interactions, thanks to the different fibronectin printed structures, and the control over the FGF-2 dosage and presentation, thanks to the easy customizable microbeads suspension loaded. Variance in all different parameters resulted in a different effect over cell colonies proliferation and survival, showcasing the capabilities of our platform to adapt for the study of different cell contexts.

FGER-1 Expression on Direct Stimulated hHF-MSCs

The stimulation of MSCs with FGF-2 not only enhances their proliferation rate but also produces changes in the expression of several proteins and membrane receptors. It has been described that FGF-2 treatment on MSCs regulates the expression of several genes such as Twist2, Spry4 or Pparg among others. FGF-2 has also showed to alter the expression of the FGF receptor family, upregulating the expression of FGF receptor 1 and 4 and suppressing the expression of FGF receptors 2 and 3. One of the receptors that is overexpressed in FGF-2 treated MSCs, the FGF receptor 1 (FGFR-1), is a tyrosine kinase receptor that participates in a number of cell processes such as bone formation and regeneration in humans. In the case of MSCs treated with FGF-2, the enhanced presence of the receptor can found in the cells membrane even after 4 hours of treatment.

To test if the co-pattern between hHF-MSCs and FGF-2-functionalized microbeads could work for the stimulation and analysis of cells in a molecular level, our hHF-MSCs were co-patterned in D₁₀₀ substrates with either 0% FGF-2 or 50% FGF-2 microbeads, and were left incubating for 6 hours. To analyze the FGFR-1, both 0% FGF-2 and 50% FGF-2 patterns were fixed and immunolabeled first with a rabbit anti-FGFR-1 antibody and then with a goat anti-rabbit Alexa Fluor 647 antibody. Fluorescence intensity of 30 different cell colonies were measured in three separated arrays.

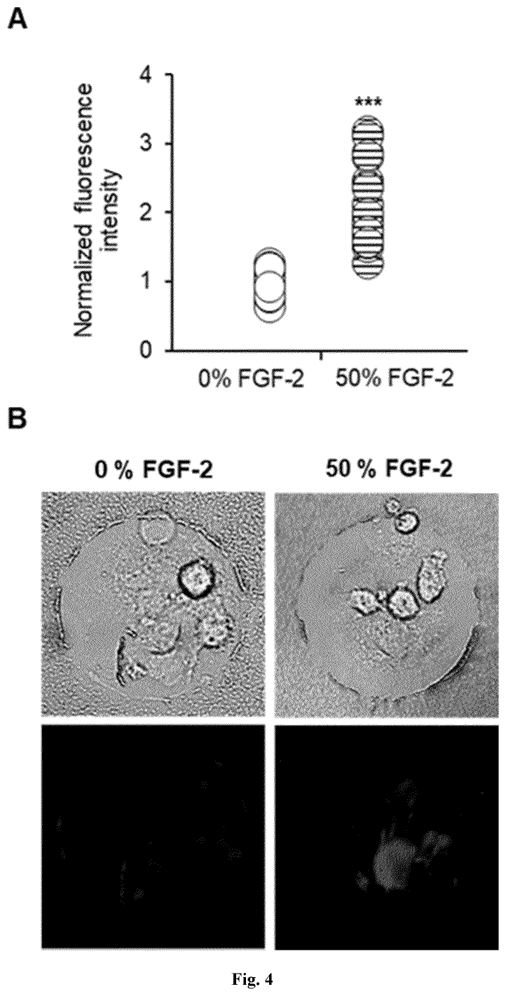

As seen in FIG. 4, the hHF-MSCs small cell-colonies patterned with 50% FGF-2 showed higher overall fluorescence intensity than those patterned with 0% FGF-2, which correlates with the quantity of FGFR-1 in the cells, validating the effect of FGF-2 in the FGFR-1 expression.

Overall, the FGFR-1 expression was doubled on the treated samples (109±47% higher fluorescence intensity, mean value) than the on non-treated samples. When the same procedure was carried out in cells cultured normally in well-plates with diluted FGF-2 10 ng mL-1, no fluorescence intensity could be observed and no differences could be appreciated between treated and untreated cells. This demonstrates that the co-patterning with FGF-2 modified microbeads increased the effect of the growth factor in the expression of FGFR-1 by the efficient presentation of the growth factor to the cells.

Thanks to the array of small cell-colonies, each dot can be analyzed as its own experimental scenario. We can see that the upregulation of FGFR-1 in our hHF-MSCs ranged from 50% to 200% when compared to non-treated cells. This also demonstrates the capability of our platform for the monitoring of biochemical changes in cells surface.

Direct Stimulation of Cell Proliferation on Patterned hHF-MSCs.

Through the combined pattern of hHF-MSC small cell-colonies and FGF-2 functionalized microbeads, spatial control and solid phase-presentation of the growth factor was achieved. This allowed the controlled stimulation of the small cell-colonies.

The number of cells in each small cell-colony after 24 h for both type of cell pattern tested (D₅₀ and D₁₀₀) was studied using two combinations of microbeads (0% FGF-2 and 50% FGF-2).

For all cases in which cells were presented with FGF-2 (50% FGF-2 patterns), higher number of cells per dot could be observed, confirming the stimulation of patterned hHF-MSCs.

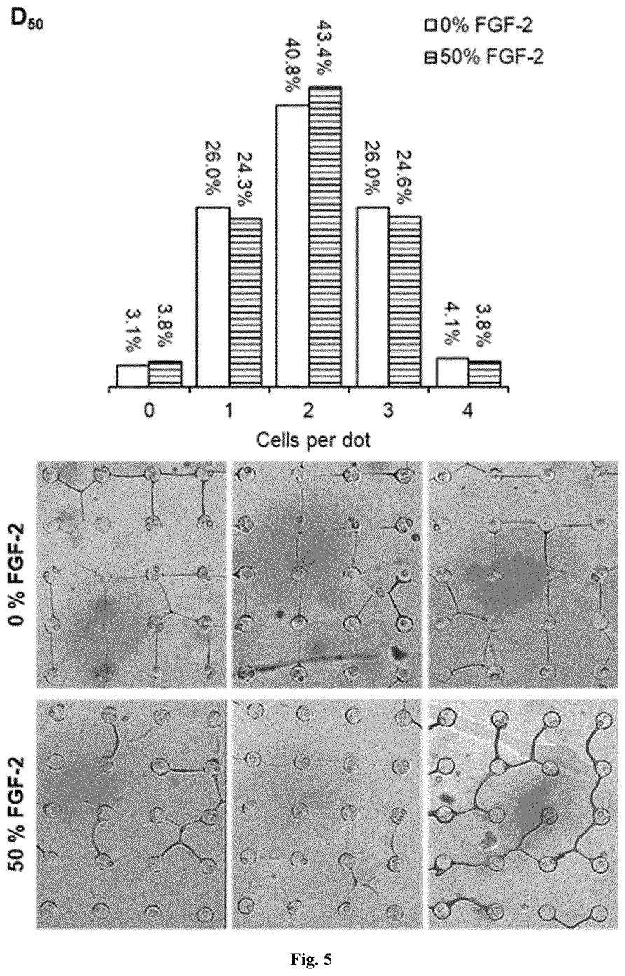

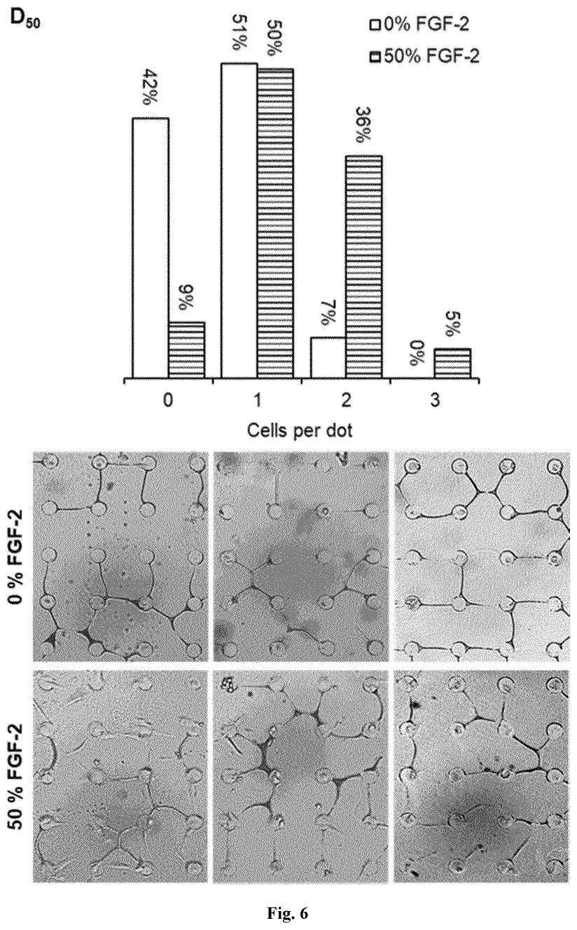

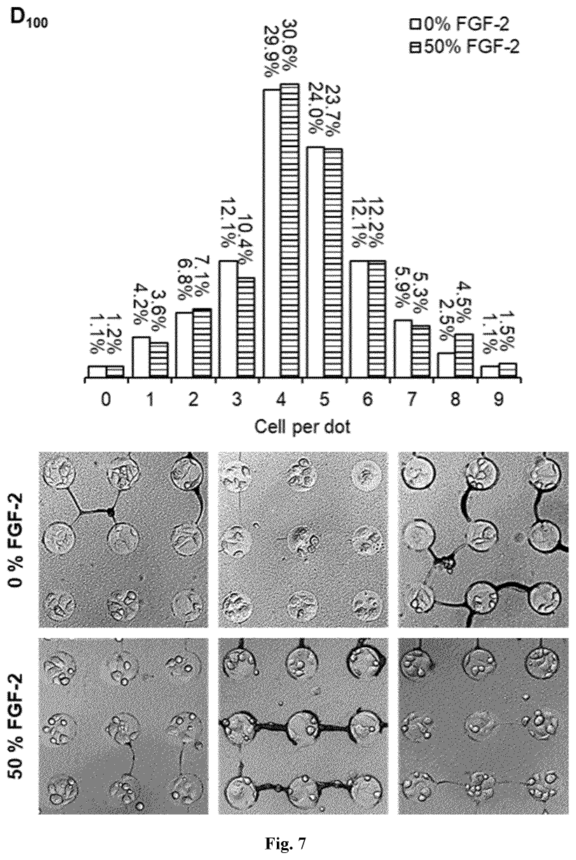

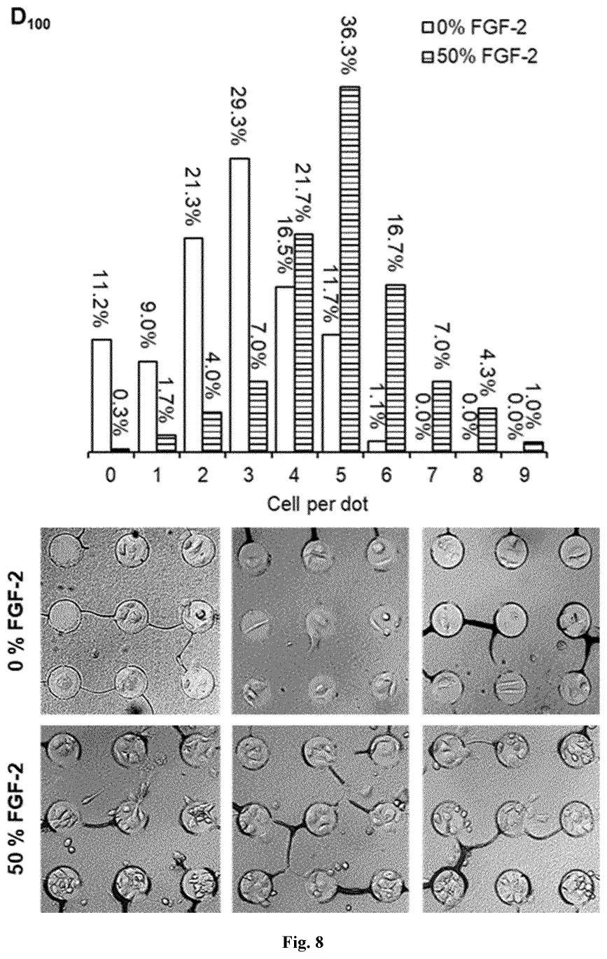

FIG. 5 (0 h) and FIG. 6 (24 h), show the change in the number of cells per dot in the case of the D₅₀ patterns. FIG. 7 (0 h) and FIG. 8 (24 h) show the change in the number of cells per dot in the case of the D₁₀₀ patterns.

FGF2-Driven Stimulation of hHF-MSCs

To address the response of hHF-MSCs to FGF-2, we analyzed the proliferation of our MSCs on regular cultures both with diluted FGF-2 and with wells treated with FGF-2. As the array of cells required serum free medium (SFM), the effect of FGF was tested both in cells cultured with SFM and with Complete Medium (CM). hHF-MSCs (p4) were cultured with CM in t75 flaks. Cells were maintained in culture until reaching 80% confluence for all experiments. 500 μL of cell suspension 105 cells mL⁻¹ were cultured in 24-well cell culture plates with three different types of medium: SFM, SFM+FGF-2 (10 ng mL⁻¹), CM and CM+FGF-2 (10 ng mL⁻¹). Three samples were studied per experimental condition. Cells were left in the incubator for 48 hours. At the end of the assay, cells were photographed and counted.

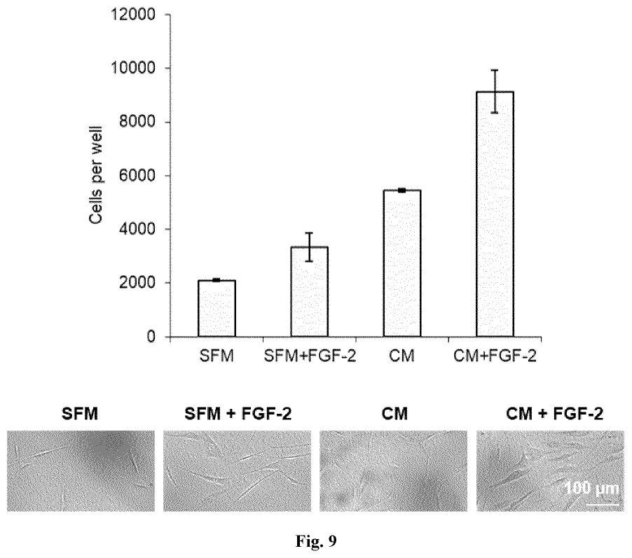

As seen in FIG. 9, in the case of cells incubated in SFM, there was up to 58% more cells in the wells treated with FGF than those left untreated (2133±47 and 3333±518 cells/well for cells in SFM and SFM+FGF 10 ng/mL, respectively). For cells in the CM, the differences observed were even higher, as there was up to 67% more cells in the wells with diluted FGF than those only incubated with CM (5450±70 and 9133±801 cells per well for cells in CM and CM+FGF 1 ng mL⁻¹, respectively). It should be noted that the presence of serum (FBS) is still the major factor affecting the maintenance and proliferation of the culture due to the high number of different factors contained. Up to 159% more cells could be found in the wells incubated with CM when compared to wells only containing SFM. Additionally, cells showcased a more elongated form and higher cell area in the samples treated with FGF.

Before testing the effect of the FGF-stimulation on a cell array, we analyzed the direct stimulation through the functionalization of the wells with FGF-2. Wells of a 24-well plates were incubated with 500 μL of streptavidin solution (200 μg mL⁻¹) for one hour. Afterwards, wells were rinsed with PBS and then incubated with FGF-2 (10 ng mL⁻¹) for an additional hour. Wells were afterwards rinsed with PBS. 500 μL of cell suspension 10000 cells mL⁻¹ in SFM were then loaded in both treated and non-treated wells (n=3). Additionally, another three wells were loaded with 500 μL of cell suspension 105 cells mL⁻¹ in SFM+FGF-2 (10 ng mL⁻¹). Cells were left in the incubator for 24 hours. At the end of the assay, cells were photographed and counted.

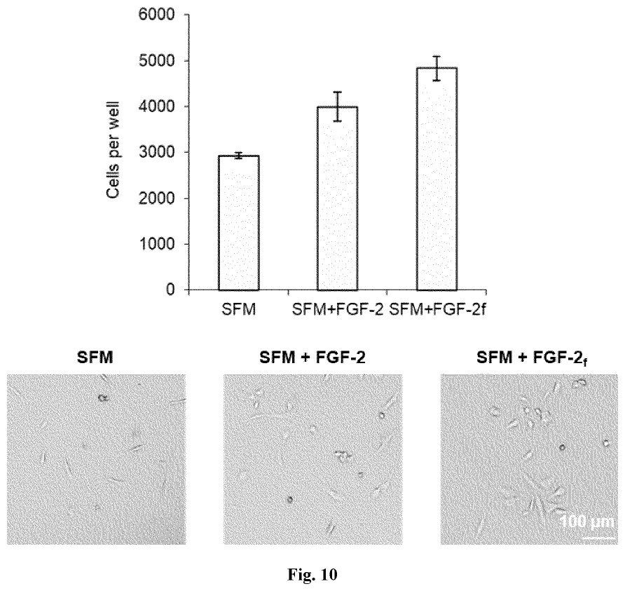

As with the case with batch cultures, in all the cases the addition of FGF resulted in an increased number of cells in the wells at the end of the experiment (see FIG. 10). Up to 31% more cells could be observed in wells with SMF+FGF when compared to cells only cultured in SFM (2877±69 and 3788±309 cells/well for cells in SFM and SFM+FGF 10 ng/mL, respectively). Comparing diluted FGF to wells functionalized with FGF, up to 29% more cells could be observed in the case of the latter (4911±258 cells/well), indicating the direct and constant interaction between the growth factor and the cells does increases the cell proliferation and culture maintenance.

The increment of cell number in samples incubated with fixed FGF-2 when compared with cells cultured without FGF-2 was similar to those obtained for diluted FGF-2 but 24 hours earlier, indicating a greater stimuli when cells are in direct contact with the growth factor. It should be noted however, that the attached cells in the functionalized wells grow in the form of patches, probably meaning that the functionalization of the surface is not homogenous.

FGF2-Driven Stimulation of hHF-MSCs Through Direct Interactions with FGF2 Functionalized Microbeads

To evaluate the FGF-2 driven stimulation through direct contact with functionalized microbeads, hHF-MSCs were directly adhered on top of microbeads patterns with or without previous functionalization with FGF-2.

For the patterning of the microbeads, 0% FGF-2 and 100% FGF-2 microbeads suspensions were loaded in empty bottom-less PDMS channels (1000×5000×13 μm, width×length×height). Fibronectin was diluted in both suspension for a final concentration of 50 μg mL⁻¹ to improve cell attachment. Vacuum lithography protocol was carried out as usual inside of PMMA wells with glass bottom. After overnight incubation and solvent evaporation, 1 mL of BSA solution 5% was loaded in all wells. Subsequently, wells were loaded with 750 μL of hHF-MSCs suspension (p4) 10⁶ cells mL⁻¹ in SFM and left inside the incubator for 2 hours on constant oscillation in a rocker (n=3 per experimental condition). Afterwards, medium was retrieved from all wells. Wells were rinsed three times with PBS and replenish with 750 μL of fresh SFM. All samples were left inside the incubator for 24 hours. At the end of the experiment, cells were photographed and counted.

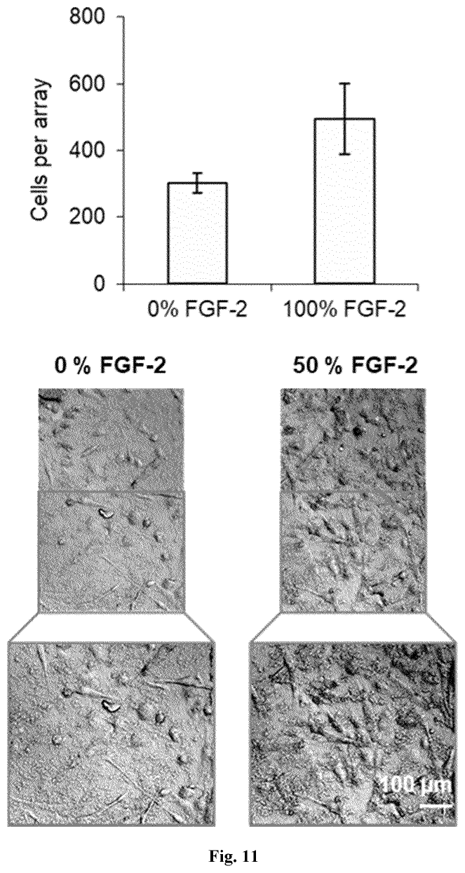

The patterning of FGF-2 functionalized microbeads on the surface allowed to control the deposition of the growth factor in the surface more efficiently than the direct functionalization of the bottom of the well itself. Once more, the presence of FGF-2 affected cell's proliferation and survival, as the number of cells in 100% FGF-2 microbeads patterns was higher than the number of cells in samples incubated with non-treated microbeads (see FIG. 11). Up to 63% more cells could be observed in the patterns containing 100% FGF microbeads only in 24 hours.

### Example 2: Method for Determining if a Cell Population Secretes to the Medium a Substance of Interest

### Example 2.1: Materials and Methods

### Materials

Polydimethylsiloxane silicone elastomer SYLGARD 184 and curing agent were purchased from Ellsworth adhesives, Spain. Streptavidin coated polystyrene microbeads (400 nm diameter) were purchased from Immunostep, Spain. VEGF, bovine 3 fibronectin, Cell culture t75 flasks, Dulbecco's modified eagle's medium (DMEM), fetal bovine serum (FBS), penicillin/streptomycin (P/S) and goat anti-mouse polyclonal IgG Alex Fluor 488 were purchased from Fisher Scientific, Spain. Bovine serum albumin (BSA) was purchased from Sigma Aldrich, Spain. Milk powder was obtained from Nativa 2, Nestle. Paraformaldehyde 4% was purchased from Panreac Quimica, Spain. Biotin goat anti-VEGF polyclonal IgG and mouse anti-VEGF monoclonal IgG were purchased from R&D Systems, USA. VEGF ELISA detection kit EA100376 was purchased from Quimigen, Spain. Human adult Mesenchymal Stromal Cells were obtained from human follicles (hHF-MCSs, p4). Polymethyl methacrylate (PMMA) Plexiglas 4 mm, was purchased from Evonik Industries AG, Germany. Pressure Sensitive Adhesive (PSA) ARcare 8939 was purchased from Adhesive Research, Ireland.

Oligonucleotides (DNA probes) were synthesized by Integrated DNA Technologies (IDT, Belgium). Firstly, an aptamer probe (Apt), containing the VEGF aptamer and a biotin on its 3′ end and the following sequence:

(SEQ ID NO: 1)
5′-TGTGGGGGTGGACTGGGTGGGTACCGTCACTCGCCTCGCACCGTC
C-Biotin-3′

Secondly, a fluorescence probe (F), partially complementary to Apt with a Cy5 fluorophore on its 3′ end and the following sequence:

(SEQ ID NO: 2)
5′-GGACGGTGCGAGGCG-Cy5Sp-3′

Finally, a Quencher probe (Q), partially complementary with an Iowa Black quencher on its 5′ end and the following sequence:

(SEQ ID NO: 3)
5′-IabRQ-GTGACGGTACCC-3′

### Solutions

VEGF capture antibody (VEGF-Ab) solution consisted of biotin polyclonal goat anti-VEGF IgG (1 μg mL⁻¹) with 0.2% milk powder (w/v) in PBS. Primary antibody (Primary Ab) solution consisted of mouse monoclonal anti-VEGF IgG (1 μg mL⁻¹), 0.2% milk powder (w/v), 1% goat serum (v/v) in PBS. Secondary antibody (Secondary Ab) solution consisted of goat anti-mouse IgG (1 μg mL⁻¹), 0.2% milk powder (w/v), 1% goat serum (v/v) in PBS. VEGF structure switching signaling aptamer (VEGF-SSSA) solution consisted on fluorescence (F), aptamer (Apt) and quencher (Q) probes 200, 600, 600 nM in PBS. Complete Medium (CM) consisted of DMEM supplemented with 30% FBS and 10% P/S. Serum-free DMEM medium (SFM) consisted of DMEM with 10% P/S.

### Fabrication of PDMS Slabs

In order to generate combined patterns of cells and microbeads, PDMS slabs with channel-like structures with pillars inside were designed and fabricated following previously published protocols (Garcia-Hernando, M.; et al., Anal. Chem. 2020, 92 (14), 9658-9665). Briefly, all structures were designed using CleWin software and adapted into several photomasks. The obtained photomasks were transferred to silicon wafers through photolithography, obtaining structures containing hills and valleys corresponding to the channels and to the pillars, respectively. The silicon wafers served as masters for the generation of the PDMS slabs. 40 mL of PDMS (silicone elastomer and curing agent ratio 10:1, respectively), were poured on top of the masters, degassed and polymerized at 70° C. for 2 h. The resulting PDMS slabs contained bottom-less channels with dimensions of 1000×5000×13 μm (width×length×height, respectively) containing small pillars of 50 and 100 μm diameter separated each other by 100 μm, named Slab₅₀ and Slab100 onwards, respectively.

### Printing and Vacuum Lithography

The patterning of adhesion protein spot surrounded by streptavidin coated polystyrene microbeads was carried out, in a single step, using the PnV Litho method. In all cases, PDMS slabs were punched twice in order to generate a 2 mm diameter inlet and a 1 mm outlet.

In order to generate the pattern of proteins, the pillars inside of the channels were wetted with a solution of fibronectin 50 μg mL⁻¹ for 30 min. Afterwards, the PDMS slabs were rinsed with distilled water and dried with compressed air. Then, the PDMS slabs were put in contact to the glass covers (24×60 mm), serving the glass as the bottom of the channels, enclosing them. Glass covers were carefully rinsed with ethanol and distilled water and were dried with compressed air before being assembled to PMMA wells (see Supporting Information 1 for specifications). The resulted pasted PDMS slabs on glass were vacuumed in a desiccator for 30 min in order to remove the air inside the PDMS matrix and promote liquid movement. Afterwards, the outlets were plugged with tape and 2 μL of microbeads suspension (8×10¹⁰ microbeads mL⁻¹) were loaded in the inlets. The outlet was needed to avoid the phase-separation produced during flow to interfere with the distribution of the suspension inside the channel. Moreover, the outlet needed to be closed during filling of the channel to maintain continuous passive pumping. The microbeads suspension was let to flow until it started filling the outlet. Tape at the outlet was removed after 5 min and the outlet was left opened for solvent evaporation at 4° C. overnight. Finally, PDMS slabs were removed and the combined fibronectin and microbead structures were revealed on the surface of the glass.

### VEGF Detection and Quantification on Patterned Microbeads

Microbeads were patterned through PnV Litho as previously explained using Slab₅₀. After solvent evaporation and PDMS removal, the microbeads patterns, in the PMMA wells, were incubated with 1 mL of milk powder (5% w/v) solution as blocking agent for 1 h. Afterwards, wells were rinsed with PBS three times and the patterns were incubated with 400 μL of either VEGF-Ab solution or the VEGF-SSSA solution for 45 min. Then, the wells were rinsed again with PBS three times. Afterwards, microbeads patterns were incubated with 400 μL of VEGF solution at different concentrations (0.001, 0.010, 0.100 and 1.000 μg mL⁻¹) with 0.2% milk powder (w/v) in PBS solution for another 45 min (n=4 samples were performed per experimental condition). Another set of wells were incubated with PBS 0.2% milk powder (w/v) serving as negative controls (n=4). In the case of the microbeads functionalized with the VEGF-SSSA, the wells were rinsed three times with PBS and images were immediately photographed in a fluorescence microscope. The samples functionalized with VEGF-Ab were rinsed three times and then incubated with 400 μL of primary Ab solution for another 45 min. Following that, the wells were rinsed three times with PBS and incubated with the secondary Ab solution for 20 min. At the end of the assay, the wells were rinsed three times with PBS and then photographed in a fluorescence microscope, see section 2.8.

Quantification of VEGF Secreted from the Supernatant of Conventional hHF-MSCs Cultures

To address the secretion of VEGF from our cell model, hHF-MSCs were cultured in CM in t75 flaks. Cells were maintained in culture until reaching 80% confluence. Afterwards, cells were detached and resuspended in either CM or SFM for a concentration of 105 cells mL⁻¹. 500 μL of the suspension of cells were loaded in a 24-well plate and were left in the incubator for 48 h. After that, supernatant was retrieved and cell were counted. VEGF secreted from the culture was measured using an ELISA kit following the commercial protocol (n=4 per experimental condition).

To validate the microbeads patterns as sensors for a real cell secretion scenario, hHF-MSCs, were cultured with CM in t75 flaks and maintained in culture until reaching 80% confluence. Afterwards, cells were detached and resuspended in CM for a concentration of 105 cells mL⁻¹. 500 μL of the suspension of the cells was loaded in a 24-well plate and were left in the incubator for 48 h. After that, supernatant was retrieved and cells were counted.

VEGF-Ab and VEGF-SSSA functionalized microbeads patterns were prepared through vacuum-driven soft lithography as previously explained using Slabs₅₀. The patterns were loaded with 400 μL of the retrieved supernatant and were left incubating for 45 min (n=3 per experimental condition). In another experiment, patterns were just loaded with CM as negative control (n=3). The VEGF-SSSA functionalized patterns were rinsed three times with PBS and immediately observed and imaged. The VEGF-Ab functionalized microbeads patterns were rinsed and incubated with primary Ab and secondary Ab as explained before and then the results were observed and imaged.

Direct Monitoring of VEGF Secretion of Patterned hHF-MSCs

Combined patterns of microbeads and fibronectin were prepared through PnV Litho as previously explained using the Slab₁₀₀. Wells were loaded with 1 mL of a solution of 5% BSA (w/v) and 5% milk powder (w/v) as a blocking agent for 1 h. Microbeads patterns were functionalized with VEGF-Ab and VEGF-SSSA, as previously explained.

hHF-MSCs (p4) were detached from the flasks and were resuspended in SFM at a concentration of 105 cells mL⁻¹. 750 μL of the cell suspension was loaded in the samples and incubated on constant oscillation in a in a Vari-Mix steep angle rocker (Thermo Fisher, Spain) inside an incubator at 37° C. and 5% CO₂ air atmosphere for 120 min (n=3 per experimental condition). Another set of samples were loaded with SFM serving a negative controls (n=3). Afterwards, medium was retrieved, the wells were rinsed three times with PBS to wash out any non-attached cells and were loaded with SFM medium and left in the incubator for 48 h. Movement and vibration of the wells was avoided during these intervals. At the end of the assay, hHF-MSCs colonies with VEGF-SSSA functionalized microbeads patterns were rinsed three times with PBS and fixed with paraformaldehyde 4% for 5 min. Then, brightfield and fluorescence microscopy images were taken, see section 2.8. For the samples with VEGF-Ab functionalized patterns, wells were rinsed three times and cells were fixed with paraformaldehyde 4% for 5 min. Afterwards, wells were incubated with primary Ab and secondary Ab solutions, as previously explained, and brightfield and fluorescence microscopy images were taken for analysis of the results.

### Image and Data Analysis

Brightfield and fluorescence microscope images were taken with a modified Nikon Eclipse TE2000-S inverted microscope (USA), with and adapted Andor Zyla sCMOS black and white camera (Oxford Instruments, UK). Lumencor laser 640 nm was used as light source for excitation and Quad EM filter: 446/523/600/677 with 4 TM bands: 446/34+523/42+600/36+677/28. Microscopy images were processed by NIS and FiJi/ImageJ software.

Normalize Fluorescence Intensity was calculated through the normalization of each value to the mean value or their respective negative control, following equation 1:

$$Normalized Fluoresence Intensity = \frac{Fluorescence Intensity Sample}{Mean Fluorescence Intensity Negative Controls} \qquad ( eq . 1 )$$

For the analysis of the fluorescence intensity in the immediacy of each spot, a circular ROI was used that covered from the edge of the spot up to 10 μm away from it.

For the analysis of the diffusion of the fluorescence intensity, a rectangular ROI with a width of 5 μm was used, and the fluorescence intensity was measured every 5 μm. To ensure proper and homogenous data recollection and analysis of the direct detection of secreted VEGF from patterned small cell-colonies, a selection criteria was used. At least one cell must remain in the pattern by the end of the assay. Moreover, the distribution of the microbeads surrounding each spot has to be similar and homogeneous in both samples and negative controls.

To calculate cell secretion from the concentration obtained in the cell patterns, we designated the volume corresponding to each small cell-colony. This is an approximation, since further work is required to properly calculate the volume in which it is possible to observe the secreted VEGF diffused from each cell-colony.

### Example 2.2: Results and Discussion

### Microbeads as Biosensors

Streptavidin coated microbeads were chosen as the sensing probes for this work, due to their inert nature and their good interaction with the glass substrate upon solvent evaporation. The streptavidin coating allowed the functionalization of the microbeads with biotinylated molecules in a versatile way due to the strong bond between streptavidin and biotin.

For the monitoring of cell secretion, VEGF was chosen as secretion model. VEGF is a cell secreted inducer of angiogenesis and vasculogenesis, and is tightly related to several diseases including cancer and regenerative disorders, becoming a key factor in diagnosis and prognosis.

Two different models were tested as biosensors for the monitoring of VEGF secretion. First, a conventional sandwich immunoassay (VEGF-Ab) was adapted, using a commercial well-known biotinylated anti-VEGF antibody as the capture antibody for the functionalization of the microbeads and the detection of the secreted VEGF. Secondly, a biotinylated SSSA (VEGF-SSSA) previously developed in our group and based on the VEGF aptamer 3R02 was also tested for the capture and label-free monitoring of the secretion of VEGF. The three-part VEGF-SSSA consisted of three probes. On its native state, the proximity between the fluorophore and the quencher, achieved through the binding of the probes, effectively quenched the fluorescence signal produced by the fluorophore. In the presence of VEGF, the VEGF-SSSA underwent a conformational change, due to the binding of Apt with the growth factor, which caused the displacement of Q from the structure and disabled the quenching effect produced on the fluorophore. This permitted to link the fluorescence intensity to the presence of VEGF.

Calibration of Sensing Microbeads with Spiked VEGF Solutions

An assay with fluorescent biotin was carried out, to test the potential of the patterned microbeads to capture biomolecules. Moreover, the assay was used to determine the capability of the fluorescence analysis method, using microscope images, for the quantification of the concentration of the captured biomolecules. Patterned microbeads were incubated with increased concentrations of fluorescent biotin solutions. As expected, the fluorescence intensity increased with biotin concentration, and this increase was quantified. The homogenous distribution of the microbeads as well as the homogenous distribution of the fluorescence in the surface confirms that the patterns of functionalized microbead can, in principle, serve as a powerful tool for biosensing.

Following the same protocol than in the previous assay, the biotin solutions were substituted by a solution of either capture VEGF-Ab, 1 μg mL⁻¹, or VEGF-SSSA 200:600:600 nM for the fluorescence (F), the aptamer (Apt) and quencher (Q) probes, respectively. Both functionalizations served as bioreceptors for the specific capture of VEGF. Afterwards, both types of functionalized microbeads patterns were incubated with different concentrations of VEGF (0.001, 0.010, 0.100 and 1.000 μg mL⁻¹).

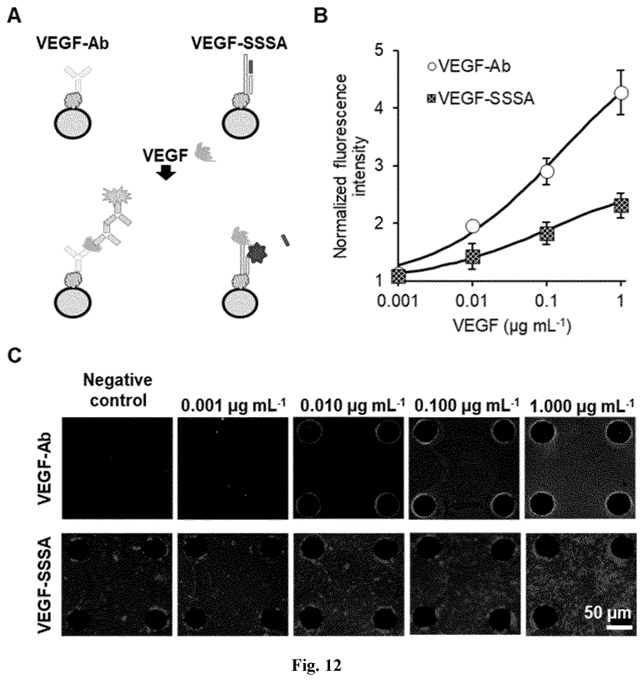

In the case of the microbeads functionalized with VEGF-SSSA, samples were directly analyzed using fluorescence microscopy. For the microbeads functionalized with VEGF-Ab, samples were incubated with primary anti-VEGF mouse antibody and fluorescent secondary goat anti-mouse antibody solutions to obtain a fluorescence signal due to the capture of the VEGF. In both cases, fluorescence intensity increased with the increase of VEGF concentrations, FIG. 12. In the case of the VEGF-Ab assay, all VEGF concentrations presented more fluorescence intensity when compared with the negative controls (from 1.12±0.03 to 4.3±0.4 normalized fluorescence intensity). The LoD was calculated following equation 2 to be 0.8 ng mL⁻¹.

$$LoD = Mean Normalized Negative Control \pm 3 SD \qquad ( eq . 2 )$$

In contrast to the VEGF-Ab assay, the microbeads functionalized with the VEGF-SSSA failed to produce any significant fluorescence intensity increment for the lowest concentration investigated (0.001 μg mL⁻¹). For the rest of the concentrations, detectable fluorescence intensities could be observed (from 1.4±0.2 to 2.4±0.1 normalized fluorescence intensity). The LoD was calculated following equation 1 to be 8.0 ng mL⁻¹.

These results confirmed the capacity of the microbeads pattern to capture and detect dissolved VEGF in both techniques. Samples prepared with VEGF-Ab showcased high sensitivity for the growth factor and low variability on the generated fluorescence intensity.

A higher fluorescence intensity was observed in the edge of each dot, due to the accumulation of microbeads. This accumulation was explained by the suction effect produced by each PDMS pillar during the fabrication of the microbeads pattern. The fluorescence intensity was highly homogeneous among samples and the normalized fluorescence intensity was constant over the different zones of the microbeads patterns investigated.

The samples prepared with VEGF-SSSA allowed the direct detection and quantification of VEGF, which gave promising insight on the capability of our platform for the real-time monitoring of cell secretion. It should be considered that, this biosensor configuration was less sensitive than the VEGF-Ab, since the fluorescence intensities for each concentration were closer to each other than in the previous scenario, and presented higher heterogeneity on the fluorescence intensity distribution over the pattern. This behavior can be explained considering that the SSSA-protein aggregates may generate over the pattern producing a residual fluorescence observable even at its quenched state.

Quantification of hHF-MSCs Secreted VEGE

Cells were incubated in conventional cell culture wells (10⁵ cells mL⁻¹) for 48 h in both SFM and complete medium 30% FBS (CM) to characterize the VEGF secretion from the hHF-MSCs. The supernatant of both samples were analyzed using a conventional commercial VEGF ELISA kit, as described in the Experimental section.

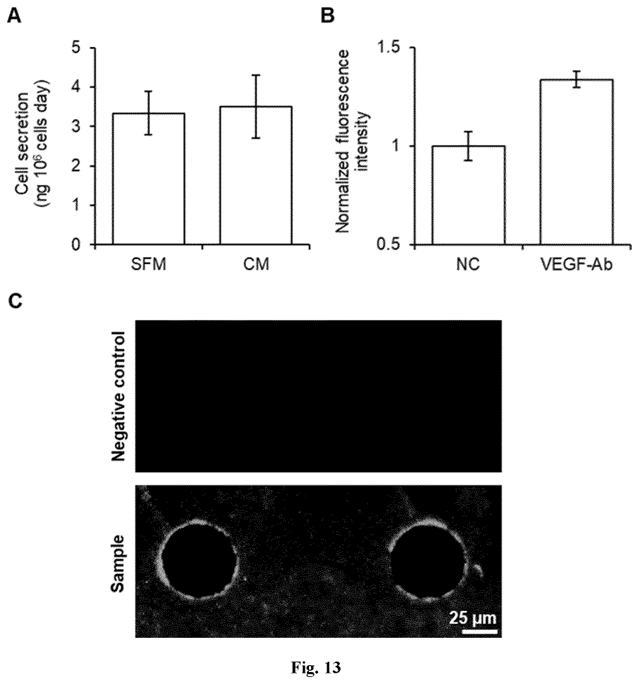

The ELISA assay, FIG. 13A, showcased that, in the case of cells cultured in CM the concentration after 48 h was 1.6±0.3 ng mL⁻¹. On the other hand, in the case of cells cultured in SFM, the concentration after 48 h was 1.1±0.2 ng mL⁻¹. This corresponded to a cell secretion in the range of 2.7-4.3 and 2.8-3.9 ng per 10⁶ cells per day for the cells cultured in CM and SFM, respectively. These values featured the range of cell secretion coming from our hHF-MSCs and indicated that there were no significant differences between culturing the cells with or without serum.

To validate the use of our platform for the analysis of cell secretion in real scenarios, hHF-MSCs were incubated in conventional cell culture wells (10⁵ cells mL⁻¹) for 48 h in CM. Then, the supernatant solution was taken from the cell culture and loaded over the microbead patterns. VEGF secretion was analyzed by fluorescence analysis of the images taken from the microbeads pattern on either the VEGF-Ab or VEGF-SSSA microbeads.

In the samples patterned with the VEGF-CAb functionalized microbeads, higher normalized fluorescence intensity, 1.38±0.04, was obtained when compared to the negative controls, FIGS. 13B and C. The increase in fluorescence intensity (40%) indicated a positive capture and recognition of the VEGF secreted by the cells. The total concentration in the supernatant was found to be of 1.7±0.4 ng mL⁻¹, corresponding to a cell-secretion in the range of 3.4-5.3 ng per 10⁶ cells per day, which lies within the values obtained for the hHF-MSCs when analyzed using a conventional ELISA kit, validating the use of microbeads patterns for the detection of cell-secreted VEGF.

In the case of the assay with VEGF-SSSA functionalized microbeads, no significant increment in the fluorescence intensity was observed. This, however, matched well with the results obtained from the VEGF-Ab assay since the total concentration of secreted VEGF after 48 h was below the limit of detection of the VEGF-SSSA assay.

Direct Monitoring of VEGF Secretion from Patterned hHF-MSCs

In order to evaluate the capacity of the combined patterns of small cell-colonies and microbeads configuration for in situ detection of cell secretion in the surroundings of each small cell-colony, hHF-MSCs were patterned on 100 μm fibronectin dots separated by 100 μm, allowing attachment of 4-5 cells per dot. The microbeads pattern was previously functionalized with either VEGF-Ab or VEGF-SSSA to test both types of assays. The combined patterns were kept for 48 h in the incubator in static mode to avoid disrupting the normal diffusion of the secreted VEGF. After 48 h, all samples were fixed using paraformaldehyde.

As in previous assays, VEGF-SSSA samples were directly observed and imaged while VEGF-Ab samples were incubated with primary and secondary antibody solutions and then, observed and imaged.

Higher fluorescence intensity was observed in all the samples containing patterned cells when compared to the negative controls (samples without patterned cells incubated in the same conditions). Moreover, the intensity was higher at the immediate proximity of each small cell-colony, decreasing when moving away from them, indicating that proper capture and recognition of VEGF was achieved.

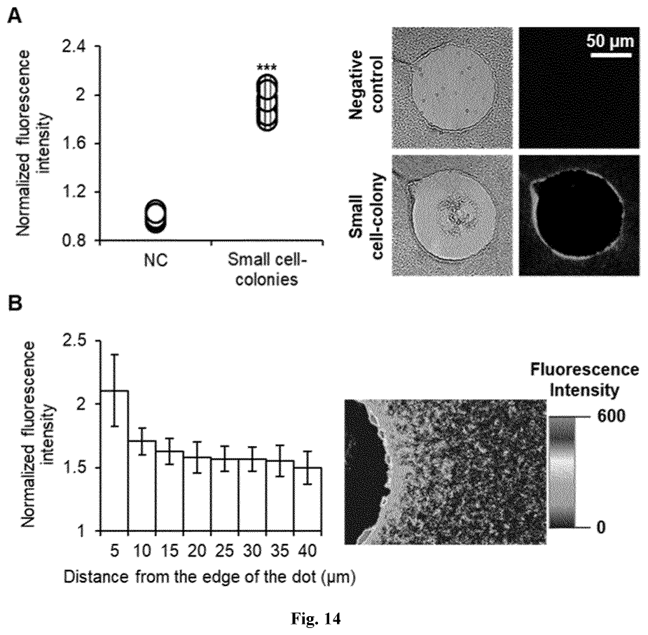

In the case of the samples functionalized with VEGF-Ab, all the analyzed cell-colonies showed a notorious increment on the fluorescence intensity in their surroundings when compared with the negative controls and a similar fluorescence intensity when compared with each other, FIG. 14. The mean fluorescence intensity value calculated was 1.98±0.09, which translates into a localized concentration of 15±5 ng mL⁻¹ and a cell secretion range of 2.5-5.0 ng per 10⁶ cells per day, which lies within the values of cell secretion of VEGF from the hHF-MSCs found in conventional cultures.

Regarding the diffusion of the VEGF, most of the growth factor was captured within the first 10 μm diameter distance from the edge of each small cell-colony, with a fluorescence intensity value decreasing as the distance from the cells increased.

These results confirmed the capabilities of our platform to capture, detect and quantify the secretion of VEGF from each small cell-colony, allowing the detection of the secretion from a high number of replicas in a single sample.

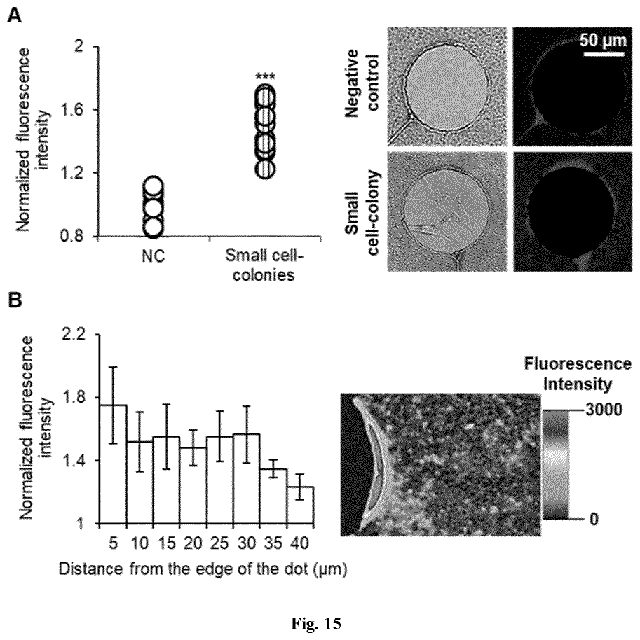

In the case of the samples with VEGF-SSSA, higher fluorescence intensity could also be observed when compared with the negative controls, indicating proper recognition of the growth factor, FIG. 15. However, greater variability in the fluorescence intensities between cell-colonies was observed than in the VEGF-Ab assay. The reason for that variability could be explained due to the SSSA and SSSA-protein aggregates formed and the residual fluorescence found in the quenched SSSA probe. The mean intensity value obtained from the samples was 1.5=0.2, which translates into a localized concentration of 19±10 ng mL⁻¹ and a cell secretion range of 2.4-9.0 ng per 10⁶ cells per day, which again correlates with the data obtained by ELISA.

The broader range was caused by the lower sensitivity of the assay and the greater heterogeneity of the samples. Nevertheless, the results still correlates with the results obtained for the VEGF-Ab assay, presenting a promising opportunity for our platform for the real-time, one-step monitoring of the cell-secretion of VEGF of a high number of replicates in a single sample. Furthermore, these results showcase that the localization of the VEGF-SSSA in the proximity of the cells enhanced its detection capabilities, as the increased localized concentration of VEGF was within the range of detection of the biosensor. This contrasts with the previous results obtained when loading cell culture supernatant, where the concentration of VEGF was below the limit of detection.

Our results confirmed that the localization of the biosensors neighboring each cell-colony greatly improved the efficiency of the detection of the cell secretion when compared to conventional methodologies. Whereas ELISA kits require the culture of thousands of cells per sample to obtain a quantifiable signal, our platform allows the detection and quantification of cell secreted VEGF from an average of 4-5 cells in, at most, 48 h. This is achieved because of the increased and localized concentration of the growth factor in the sensing area of the cell-colonies, improving the efficiency of the capture immediately after secretion.

### Example 3: Support of the Invention

### PDMS Slabs Specifications

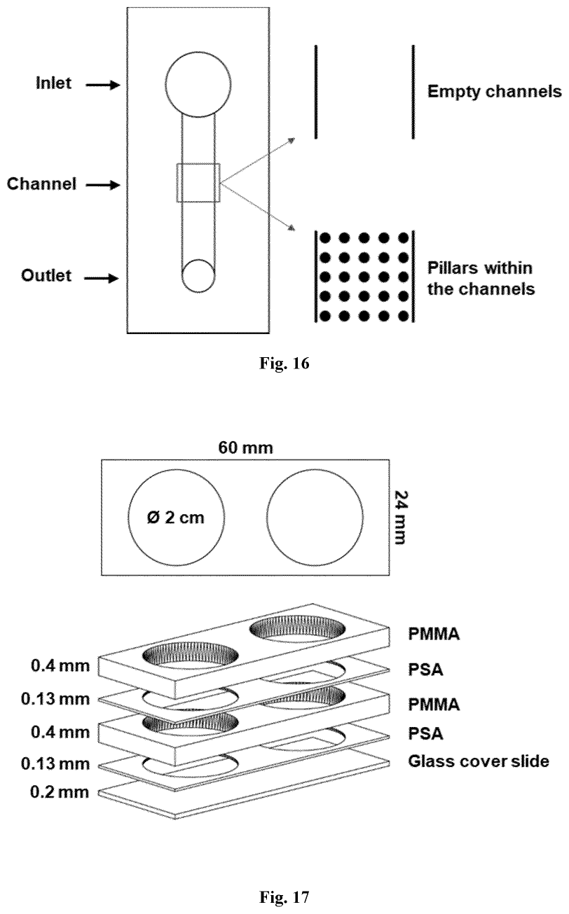

Through the vacuuming of a PDMS slab with a channel-like structure, the channel can be enclosed with a material on its bottom and tape in the outlet allowing the flow of a solution or a suspension through degas-driven flow. The channels designed for our study were either empty or contained micropillars for the printings of fibronectin dots (FIG. 16).

### PMMA Cell Culture Wells Specifications

In order to generate wells (2 cm) with a glass bottom, two polymethylmetacrylate (PMMA) layers (4 mm) were cut with a laser and pasted both between them and with the glass cover with pressure sensitive adhesive (PSA), FIG. 17.

### Printing and Vacuum Lithography

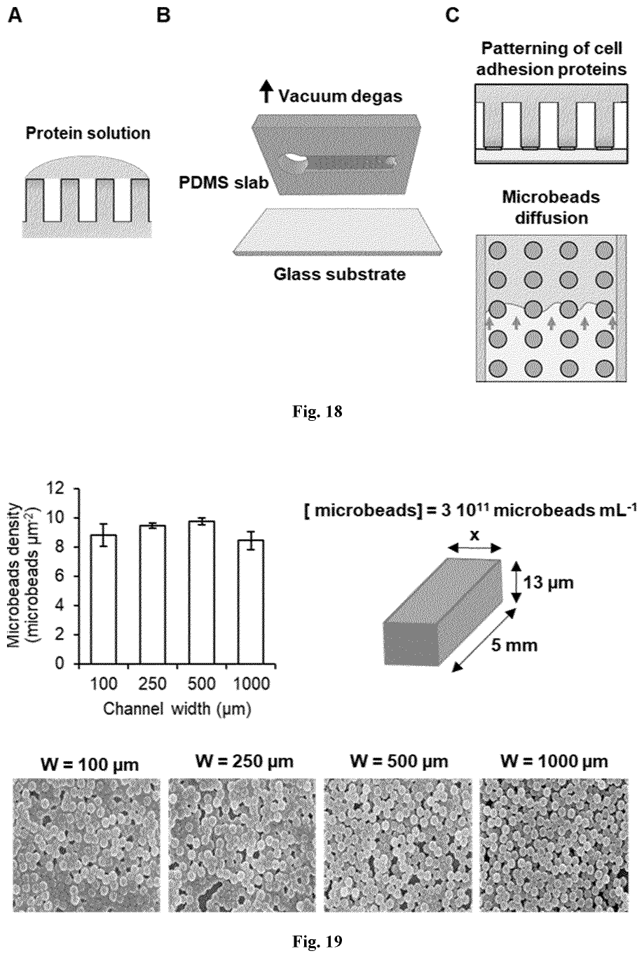

The channel-like structures containing micropillars were used for the combined patterning of cell adhesion protein dots and microbeads on the surface of glass. The micropillars were coated with a suspension of fibronectin. After retrieving the fibronectin suspension, the PDMS slabs were dried, put in intimate contact with the glass substrate and put under vacuum. The contact between the micropillars and the glass substrate allowed the dry transfer of fibronectin in the form of confined dots, while the passive pumping generated within the channel allowed the flow of the microbeads suspension, surrounding each micropillar (FIG. 18).

### Characterization of Microbeads Deposition

For the characterization of the microbeads deposition, the loading of the microbeads suspension was done through vacuum-driven lithography. An empty channel-like structure of 100×5000×13 μm (width×length×height) and a suspension concentration of 3×10¹¹ microbeads mL⁻¹ were used as a starting point for all experiments, changing only one parameter per experimental condition.

Patterning of microbeads was done on channels with changes either in their channel's width (100, 200, 500 and 1000 μm), channel's length (5000, 10000 and 20000 μm), channel's height (13 and 25 μm) or in the microbeads suspension concentration (3×10⁹, 6×10⁹, 3×10¹⁰, 6×10¹⁰, 1.5×10¹¹ and 3×10¹¹ microbeads mL⁻¹).

In the case of channel dimensions, different widths, lengths and heights were tested. In all cases, the same volume (2 μL) and the same microbeads suspension (3×10¹¹ microbeads mL⁻¹) were applied. Distribution of the microbeads after evaporation was quantified through image analysis of SEM photographs. In the case of the different widths tested (100, 200, 500 and 1000 μm), almost no difference could be observed in the microbeads deposition, indicating that the range of width studied produced a similar distribution of the suspension (see FIG. 19).

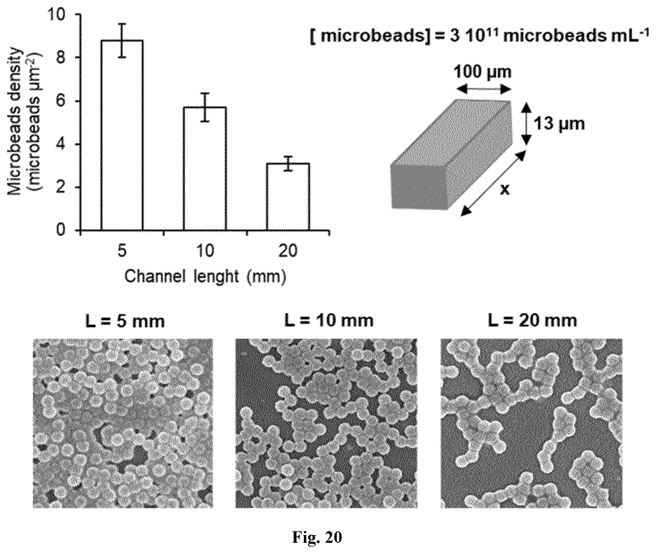

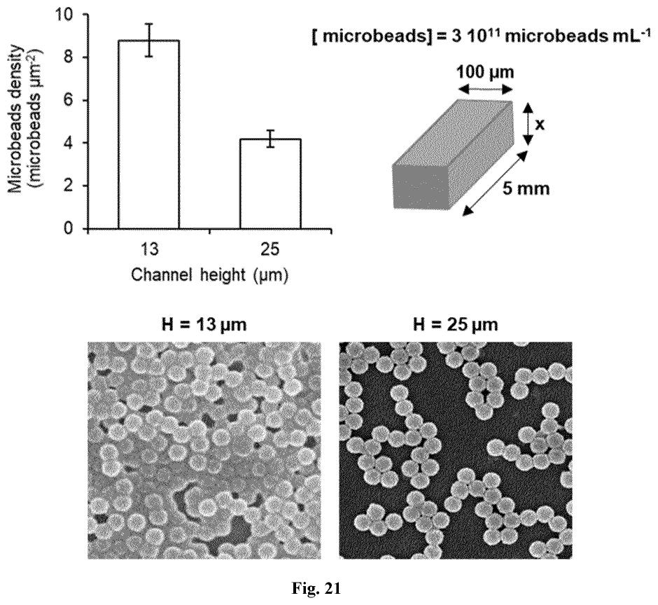

In the case of the lengths studied (5000, 10000 and 20000 μm), the density of microbeads deposited (measured as microbeads per μm²) decreased proportionally as the length of the channel increased (see FIG. 20). This implies that shorter channels concentrates the particles along the surface, whereas longer channels produce a larger distribution of the microbeads on the surface. It should be noted that the microbeads also interacts with the PDMS walls of the channel. The increased contact surface between the suspension and the PDMS in longer channels could also indicate an augmented loss of the microbeads to the PDMS.

A similar effect could be observed in the case of the different heights studied (13 and 25 μm), where higher height also produced lesser microbeads density of the surface (see FIG. 21).

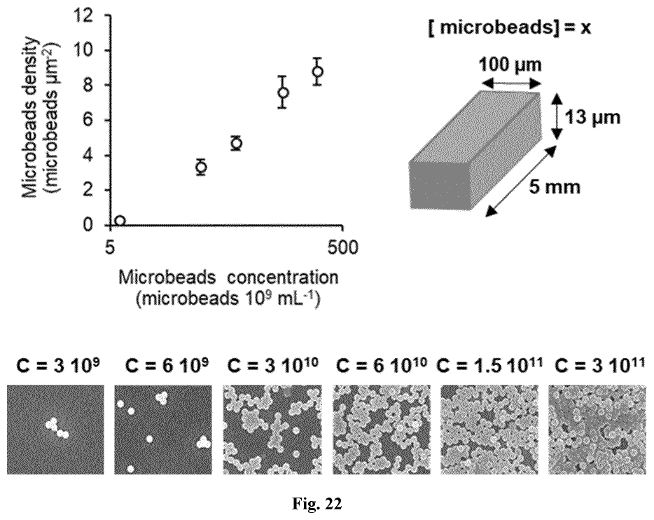

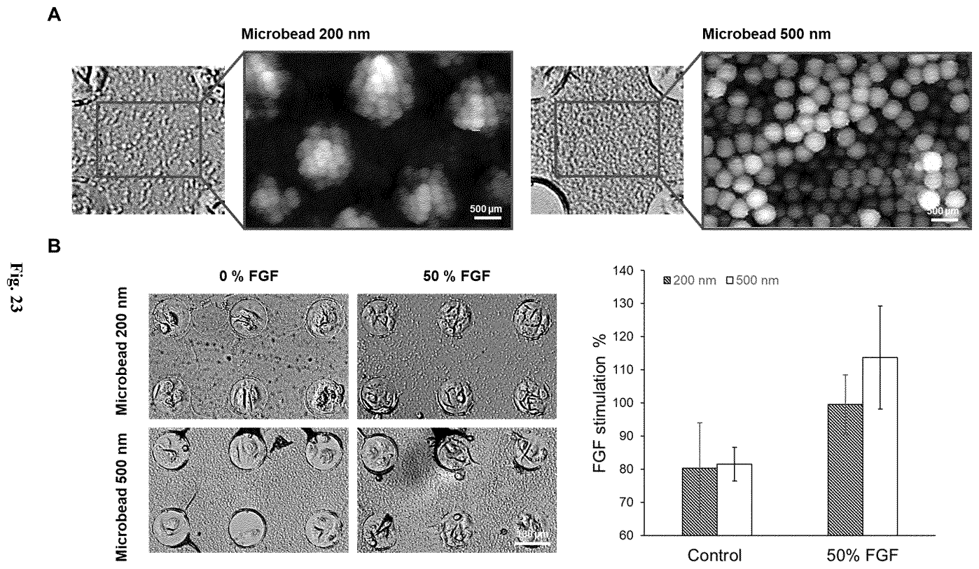

In the case of the different microbeads concentrations studied (3×10⁹, 6×10⁹, 3×10¹⁰, 6×10¹⁰, 1.5×10¹¹ and 3×10¹¹ microbeads mL⁻¹) the density of microbeads in the surface increased as expected with increased suspension concentrations (see FIG. 22). Microbeads showcased some interaction between them, being present in aggregates at low concentrations.

### Example 4: Patterning of Polystyrene Microbeads of Different Sizes

Patterns of polystyrene microbeads of different sizes (200 and 500 nm diameter) was obtained through Printing and Vacuum Lithography (PnV Litho).

PDMS slabs channel-like structures (1000×5000×13 μm, width×length x height) with pillars inside (100 μm diameter) were punched as previously explained. The pillars inside of the channels were wetted with a solution of fibronectin 50 μg mL⁻¹ for 30 min. Afterwards, PDMS slabs were rinsed with distilled water, dried with compressed air and attached to the PMMA wells with glass bottom. The resulted pasted PDMS slabs on glass were put under vacuum inside of a desiccator for 30 min. Thereafter, the outlets were plugged with tape and 2 μL of microbeads suspension (either 200 or 500 nm diameter) was loaded on the inlets. Upon solvent evaporation and PDMS removal, patterns of fibronectin dots (100 μm) surrounded by microbeads were obtained. Finally, the wells were loaded with 1 mL of BSA solution 5% as a blocking agent. As seen in FIG. 1A, patterns of both types of microbeads were obtained. While homogenous distribution of the microbeads along the pattern could be observed in both cases, the 200 nm microbeads were observed to form small accumulates, while the 500 nm microbeads formed a spread distribution from 1 to 3 layers of microbeads.

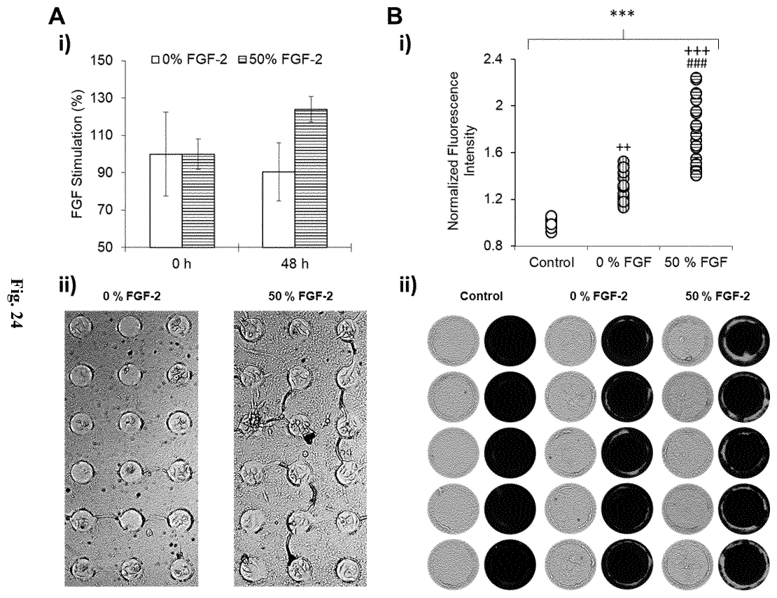

To evaluate the direct FGF-2-driven stimulation, microbeads were functionalized with FGF-2. In total, two different batch of FGF functionalized microbeads were made: one containing exclusively non-treated microbeads (0% FGF-2) and one with a mixture 1:1 of a 0% and a 100% batch (50% FGF-2). hHF-MSCs were detached from the flasks and were resuspended in serum free medium (SFM) at a concentration of 105 cells mL-1. 750 μL of the cell suspension were loaded on top of the microbeads patterns and were left inside the incubator on constant oscillation for 60 min. As seen in FIG. 23 B, patterns of hHF-MSCs and microbeads were obtained for both microbead sizes. Solid-phase presentation of FGF-2 to the patterned cells was demonstrated for both types of microbeads, as seen in the higher number of cells in all 50% FGF scenarios.

### Example 5: Combined Direct FGF-2 Stimulation and VEGF Detection on Patterned Cell Clusters

VEGF secretion from hHF-MSCs cluster patterns directly stimulated with two dosages of FGF-2 (0% FGF-2 and 50% FGF-2) was studied. In short, two types of microbeads patterns were developed, one containing 50% anti-VEGF biotin antibody functionalized microbeads and 50% blank microbeads (0% FGF-2), and another containing 50% anti-VEGF biotin antibody functionalized microbeads and 50% FGF-2 functionalized microbeads (50% FGF-2). Upon solvent evaporation and PDMS removal, patterns of fibronectin dots (100 μm) surrounded by microbeads were obtained. Finally, the wells were loaded with 1 mL of blocking solution 5% BSA, 10% casein and 1% milk powder for 2 h. hHF-MSCs were detached from the flasks and were resuspended in serum free medium (SFM) at a concentration of 105 cells mL-1. 750 μL of the cell suspension were loaded on top of the microbeads patterns and were left inside the incubator on constant oscillation for 120 min. After patterning of cells, the samples were left inside of an incubator for 48 h. Patterns without cells were also left inside the incubator as control for the secretion fluorescence. After the 48 h, cells were fixated with 4% paraformaldehyde. Samples were incubated with primary and secondary antibody solutions for VEGF detection. Brightfield and fluorescence photographs of the samples were taken at the end of the assay.

As seen in FIG. 24 A, higher number of cells could be observed in the samples containing the dosage of 50% FGF-2 after 48 h, indicating proper stimulation of the proliferation and survival of the patterned cells when directly exposed to the growth factor.

As seen in FIG. 24 B, VEGF secretion was detected in the surroundings of all small cell clusters. Higher fluorescence intensity could be observed in the surroundings of the clusters exposed to 50% FGF-2, indicating higher VEGF secretion on this scenario. This could be caused by the higher number of cells present in the 50% FGF-2 samples, a stimulation of VEGF secretion in the presence of the growth factor or by a combination of both.

In all, these results confirms the capability of the platform to simultaneously actuate and stimulate cell behaviors while detecting cell secretion, opening the door to study the effect of a particular stimulation in cells secretion.

1. A support suitable for culturing cells characterized in that it shows patterned regions, said pattern being defined by a complementary pattern of uniformly distributed microbeads attached to the support and wherein the microbeads are modified with at least one functionalizing molecule, wherein the patterned regions in the support defined by the microbeads show increased adhesive capacity to cells with respect to the regions to which the microbeads are attached.

2. The support according to claim 1 wherein the patterned regions are defined by a complementary pattern of uniformly distributed microbeads attached to the support and wherein the complementary pattern of uniformly distributed microbeads is created by allowing the microbeads to adhere to the support when the support is in contact with a stamp, said stamp having protrusions which define the pattern regions within the support, thereby preventing the microbeads from attaching to those areas of the support which are in contact with the stamp protrusions.

3. The support according to claim 1 wherein the diameter of the microbeads is between 50 nm and 25000 nm.

4. The support according to claim 3 wherein the regions in the support onto which the microbeads are attached are further coated with a compound or composition which substantially reduces cell adhesion.

5. The support according to claim 2 wherein the patterned regions are created using vacuum-driven soft lithography.

6. The support according to claim 1, wherein the microbeads are glass microbeads, polystyrene microbeads, poly (methyl methacrylate) microbeads, silica microbeads, zirconia microbeads, titanium microbeads, gold microbeads, polyethylenepolylactic acid (PLA) microbeads, poly-L-lactic acid (PLLA) microbeads, poly glycolic acid (PGA) microbeads, poly lactic-co-glycolic acid (PLGA) microbeads and poly-caprolactone (PCL) microbeads, preferably polystyrene microbeads.

7. The support according to claim 1, wherein the functionalizing molecule is attached to the microbeads through a coupling molecular pair, wherein one member of the coupling molecular pair is bound to the microbeads and the other member of the coupling molecular pair is bound to the molecule of interest.

8. The support according to claim 7 wherein the coupling molecule pair is the streptavidin-biotin molecular pair.

9. The support according to claim 1 containing at least two types of microbeads wherein each type of microbead is modified with at least one functionalizing molecule, wherein the functionalizing molecules are identical or different between the different types of microbeads.

10. The support according to claim 1, wherein the patterned regions in the support are coated with a compound or composition that promotes cell adhesion.

11. The support according to claim 10 wherein the coating of the support with a compound or composition that promotes cell adhesion is carried out by coating the protrusions of the stamp with said compound or composition that promotes cell adhesion and contacting the support with the stamp thereby allowing the transfer of the compound or composition from the stamp onto the support with a pattern that matches the pattern of protrusions of the stamp.

12. The support according to claim 10, wherein the compound that promotes cell adhesion is an extracellular matrix molecule, preferably fibronectin.

13. The support according to claim 1, where in the support is made from a material selected from a group consisting of: glass, borosilicate glass, quartz glass, polystyrene, polymethylmethacrylate (PMMA), polycarbonate, polyethylene and cyclic olefin copolymers.

14. The support according to claim 1, wherein the patterned regions have a geometrical form, preferably a circular form with a diameter of between 10 μm to 200 μm.

15. The support according to claim 1, wherein the support further contains viable cells which are attached to the support or to molecules within the support which promote cell adhesion.

16-30. (canceled)

31. A method for determining the effect of a molecule on a cell population which comprises the steps of:
(i) Providing a support according to claim 1, wherein said support contains the cell population wherein the cells of the population are attached to the support and wherein the molecule, the effect of which on the cell population is to be determined is the functionalizing molecule;
(ii) Maintaining the support under adequate conditions to allow the functionalizing molecule to exert its effect on the cells of the cell population; and
(iii) Detecting the response of the cells within the cell population to said functionalizing molecule,
wherein the detection of a response of the cells to the functionalizing molecule is indicative that the functionalizing molecule exerts an effect on the cells of the cell population.

32. The method according to claim 31 wherein the functionalizing molecule is selected from a group consisting of growth factors, differentiation factors, cell adhesion molecules or proteins, pharmaceutical small molecules and any combination thereof.

33. The method according to claim 32 wherein the growth factor is selected from the group consisting of: vascular endothelial growth factor (VEGF), collagen, bone morphogenic factor-β, epidermal cell growth factor (EGF), platelet derived growth factor (PDGF), nerve growth factor (NGF), fibroblast growth factor (FGF), insulin-like growth factor (IGF), transforming growth factor (TGF) and any combination thereof.

34. A method for determining if a cell population secretes to the medium a substance of interest which comprises the steps of:
(i) Providing a support according to claim 1, wherein said support contains a cell population wherein the cells of the population are attached to the support and wherein the functionalizing molecule comprises a molecule which is capable of interacting with the substance of interest, said interaction resulting in a detectable signal;
(ii) Culturing the cells in the adequate conditions to allow binding of the substances of interest secreted to the medium to the functionalizing molecule; and
(iii) Detecting the binding of the substance of interest to the molecule of interest by detecting the signal resulting from said interaction.

35. The method according to claim 34 wherein the substance of interest of step (i) is selected from a group consisting of: exosomes, extracellular vesicles, lipoproteins, ferritin, viruses, human growth hormone, N-methionyl human growth hormone, bovine growth hormone, parathyroid hormone, thyroxine, insulin, proinsulin, relaxin, prorelaxin, glycoprotein, follicle stimulating hormone (FSH), thyroid stimulating hormone (TSH), luteinizing hormone (LH), hepatocyte growth factor, fibroblast growth factor, prolactin, placental lactogen, factor-α, tumor growth-β, Mullerian inhibiting substance, gonadotropin-associated peptide mouse, inhibin, activing, vascular endothelial growth factor, integrin, thrombopoietin (TPO), EGF-β growth, platelet growth factor, transforming growth factor (TGF)-α, TGF-β, insulin-like factor-I growth, insulin-like factor-II growth, Erythropoietin (EPO), osteoinductive factor, interferon-α, interferon-β, interferon-γ, macrophage-CSF (M-CSF), CSF granulocyte-macrophage (GM-CSF), granulocyte-CSF (G-CSF) interleukin (IL)-1, IL-la, IL-2, IL-3, IL-4, IL-5, IL-6, IL-7, IL-8, IL-9, IL-11, IL-12; tumor necrosis factor (TNF)-α, TNF-β, leukemia inhibitory factor (LIF), kit ligand (KL), glucose, lactate, oxygen (O₂), calcium (Ca²⁺), carbon dioxide (CO₂), Hydrogen peroxide (H₂O₂), sodium (Na⁺), potassium (K⁺), cadmium (Cd²⁺), cobalt (Co²⁺), palladium (Pb²⁺), copper (Cu²⁺), silver (Ag⁺), magnesium (Mg²⁺), manganese (Mn²⁺), nickel (Ni²⁺), zinc (Zn²⁺), chromium (Cr³⁺), iron (Fe³⁺), aluminum (Al³⁺), mercury (Hg²⁺) and any combination thereof.

36. A method for determining the effect of an effector molecule on a cell population and for determining if the cell population secretes to the medium a substance of interest which comprises the steps of:
(i) Providing a support according to claim 1, wherein said support contains the cell population, wherein the cells of the population are attached to the support and wherein the first and second type of microbeads contain, respectively, a first type of functionalizing molecule which is the effector molecule and a second type of functionalizing molecule which is capable of interacting with the substance of interest, said interaction resulting in a detectable signal;
(ii) Culturing the cells under conditions adequate to allow the effector molecule to exert its effect on the cells of the cell population and the binding of the substances of interest secreted to the medium to the detector molecule; and
(iii) Detecting the response of the cells within the cell population to said effector molecule and detecting the binding of the substance of interest to the detector molecule by detecting the signal resulting from said interaction.

37. The method according to claim 36 wherein the support contains at least two types of the first type of microbead and at least two types of the second type of microbeads, wherein each type of microbead of the first type is modified with at least one functionalizing molecule, and each type of microbead of the second type is modified with at least one functionalizing molecule, wherein the functionalizing molecules are identical or different between the different types of microbeads.

38. The method according to claim 36, wherein the effector molecule is selected from a group consisting of growth factors, differentiation factors, cell adhesion molecules or proteins, pharmaceutical small molecules and any combination thereof.

39. The method according to claim 38 wherein the growth factor is selected from the group consisting of: vascular endothelial growth factor (VEGF), collagen, bone morphogenic factor-β, epidermal cell growth factor (EGF), platelet derived growth factor (PDGF), nerve growth factor (NGF), fibroblast growth factor (FGF), insulin-like growth factor (IGF), transforming growth factor (TGF) and any combination thereof.

40. The method according to claim 36, wherein the substance of interest of step (i) is selected from a group consisting of: exosomes, extracellular vesicles, lipoproteins, ferritin, viruses, human growth hormone, N-methionyl human growth hormone, bovine growth hormone, parathyroid hormone, thyroxine, insulin, proinsulin, relaxin, prorelaxin, glycoprotein, follicle stimulating hormone (FSH), thyroid stimulating hormone (TSH), luteinizing hormone (LH), hepatocyte growth factor, fibroblast growth factor, prolactin, placental lactogen, factor-α, tumor growth-β, Mullerian inhibiting substance, gonadotropin-associated peptide mouse, inhibin, activing, vascular endothelial growth factor, integrin, thrombopoietin (TPO), EGF-β growth, platelet growth factor, transforming growth factor (TGF)-α, TGF-β, insulin-like factor-I growth, insulin-like factor-II growth, Erythropoietin (EPO), osteoinductive factor, interferon-α, interferon-β, interferon-γ, macrophage-CSF (M-CSF), CSF granulocyte-macrophage (GM-CSF), granulocyte-CSF (G-CSF) interleukin (IL)-1, IL-la, IL-2, IL-3, IL-4, IL-5, IL-6, IL-7, IL-8, IL-9, IL-11, IL-12; tumor necrosis factor (TNF)-α, TNF-β, leukemia inhibitory factor (LIF), kit ligand (KL), glucose, lactate, oxygen (O₂), calcium (Ca²⁺), carbon dioxide (CO₂), Hydrogen peroxide (H₂O₂), sodium (Na⁺), potassium (K⁺), cadmium (Cd²⁺), cobalt (Co²⁺), palladium (Pb²⁺), copper (Cu²⁺), silver (Ag⁺), magnesium (Mg²⁺), manganese (Mn²⁺), nickel (Ni²⁺), zinc (Zn²⁺), chromium (Cr³⁺), iron (Fe³⁺), aluminum (Al³⁺), mercury (Hg²⁺) and any combination thereof.

41. The method according to claim 36, wherein the effect of the effector molecule on the cell population is the secretion of the substance of interest.


SEQUENCE LISTING (3 sequences)

SEQ ID NO: 1 - DNA, 46 bp, synthetic construct
  misc_feature 1..46: note = Aptamer probe
  source 1..46: mol_type = other DNA
tgtgggggtggactgggtgggtaccgtcactcgcctcgcaccgtcc

SEQ ID NO: 2 - DNA, 15 bp, synthetic construct
  misc_feature 1..15: note = Fluorescence Probe
  source 1..15: mol_type = other DNA
ggacggtgcgaggcg

SEQ ID NO: 3 - DNA, 12 bp, synthetic construct
  misc_feature 1..12: note = Quencher probe
  source 1..12: mol_type = other DNA
gtgacggtaccc